(19) United States
(12) Patent Application Publication
(10) Pub. No.: US 2010/0145386 A1
(43) Pub. Date: Jun. 10, 2010

(54) IMPLANTABLE SUPPORT DEVICE AND METHOD OF USE

(75) Inventors: E. Skott GREENHALGH, Lower Gwynedd, PA (US); John-Paul ROMANO, Chalfont, PA (US)

Correspondence Address: LEVINE BAGADE HAN LLP, 2400 GENG ROAD, SUITE 120, PALO ALTO, CA, 94303, US

(73) Assignee: STOUT MEDICAL, INC., Perkasie, PA (US)

(21) Appl. No.: 12/709,155

(22) Filed: Feb. 19, 2010

Related U.S. Application Data
(63) Continuation of application No. PCT/US2008/077102, filed on Sep. 19, 2008.
(60) Provisional application No. 60/973,702, filed on Sep. 19, 2007.

(51) Int. Cl. A61B 17/70; A61B 17/88

(52) U.S. Cl. 606/246; 606/279

(57) ABSTRACT

A plastically-deformable spinal stabilization plate is disclosed. The spinal stabilization plate can have a number of screw-holding sockets to facilitate attachment to spinal vertebra. The spinal stabilization plate can be provided with connection points to facilitate controlled deformation or transformation of the plate by use of a deformation tool. A deformation tool is also disclosed for use in transforming the plate. The spinal stabilization plate can have a deformation area with deformation struts and apertures to facilitate a controlled deformation in a predetermined direction in response to particular deformation forces.

### CROSS-REFERENCE TO RELATED APPLICATIONS

This application is a continuation of PCT Application No. PCT/US2008/077102, filed 19 Sep. 2008, which claims the benefit to U.S. Provisional Application No. 60/973,702, filed 19 Sep. 2007, which are both incorporated herein by reference in their entireties.

### BACKGROUND OF THE INVENTION

1. Field of the Invention

This invention relates to devices for providing support for biological tissue, for example to repair damaged vertebra, and methods of using the same.

2. Description of the Related Art

The spinal column is susceptible to many kinds of injuries, disease, and trauma. Some examples are herniated disks, spinal stenosis, scoliosis, fractures (e.g., compression), or a dislocation of vertebrae of the spinal column. Many of these problems may be treatable through surgical procedures. Spinal stabilization plates or assemblies of rods and clamps may be used to provide support and proper alignment to the individual vertebra as a part of these surgical procedures. Spinal plates and assemblies of rods and clamps may also be used to immobilize (e.g., to fuse) adjacent vertebra with respect to one another. Genetic irregularities, dramatic injuries, repetitive stress injuries, and disease can each result in spinal pathologies that can be treated by the use of a spinal plate device to stabilize or immobilize one or more vertebrae.

Spinal stabilization plates generally have predrilled holes to allow mounting of the plates to the individual vertebra by use of bone screws. For example, some vertebral stabilization plates attach to individual vertebra providing a stabilizing function. However, the holes in plates of this type may not align optimally with acceptable attachment points on the individual vertebra. This is a natural result of the wide variety of patients and the variation in spinal morphology among them. The inability to individually tailor the plate geometry to match the optimum vertebra attachment points can reduce the effectiveness of the treatment procedure and lead to complications if a plate is mismatched during deployment.

Because of the large number of variations in the stabilization or immobilization requirements indicated by a particular treatable condition, a spinal plate that is adjustable is desirable. This allows the surgeon to tailor the stabilization structure carefully to create the most effective stabilization arrangement and to adjust the plate for patients of different size, age, conditions, and needs.

Bone fixation devices that are adjustable in length are also known. These designs typically have plates with slidably connectable or rotatable elements. For example, some spinal stabilization plates have two longitudinally slidably interconnected attachment elements. Designs of this type typically have a larger profile than nonadjustable designs due to their increased complexity and the necessity of a slidable interface for interconnecting the multiple elements of the device. In such designs, one element of the device typically has a profile large enough to receivably accommodate or slidably interconnect with another element of the device, necessarily requiring a larger profile. The larger profile can result in discomfort for the patient and additional damage to surrounding tissue. In addition, adjustable devices can necessitate the incorporation of an additional locking or securing mechanism or configuration to ensure that the interconnected device elements retain their adjusted configuration after the surgery. These additional locking or fixation mechanisms can further increase the bulkiness of the instrumentation.

Furthermore, some spinal fixation devices fix screws in the spine and to the static fixator. The lack of motion in the fixator and the screw can cause increased pressure points at the interface of the screws and bone and at the interface of the screws and the fixators, even for some toggling variations of screws. These increased stress or pressure points can cause the bone to deform (e.g., windshield wiper effect) which can loosen the screw from the bone, and/or the screw to break.

Thus, there is a need for a spinal stabilization plate that is highly adjustable to be adaptable to a broad range of patients, while simultaneously maintaining a small profile. There is also a need for an adjustable spinal plate that can firmly retain its post-adjustment geometry without bulky fixation or locking elements. A need also exists for a spinal fixation plane that allows for relative motion between vertebrae after implantation of the plate.

### BRIEF SUMMARY OF THE INVENTION

An implantable support device and methods of using the same are disclosed. The system includes a dynamically adjustable bone fixation plate. The system can be used as an aid in vertebral or other bone fusion and stabilization of the spine or other bones. The plate can be configured before, during, or after implantation. The configuration of the plates can be transformed once or many times. The plate can be resiliently or plastically deformable. The plate can have one, two, three, or unlimited degrees of freedom for deformation. Delivery devices for removably carrying the plate to the treatment site and for adjusting the plate are also disclosed.

The plate can have a first section and a second section. The first section can be slidably attached to the second section. The first section and the second section can be separately attached to bones. The first section and the second section can be attached to provide resistance, but not complete impediment, to relative motion between the first section and the second section, or the plate can be configured so the first section and the second section can be attached to provide a substantially complete impediment to relative motion between the first section and the second section.

The plate can have an adjustable configuration. The plate can be used with cams. The cams can be placed within the plate. The cams can be rotated to alter the configuration of the plate. For example, rotating the cams can deform the struts of the plate to longitudinally lengthen or shorten the plate.

The plate can have internal ratcheting or pairs of teeth. The teeth pairs can engage each other during use (i.e., a first set of teeth can engage a second set of teeth). The engaged teeth can provide a resistive force to compression and/or expansion of the plate.

The surgeon can use the dynamic capabilities of the plate to properly size the plate for the patient (e.g., by lengthening, shortening, twisting, or combinations thereof to match patient anatomy). The dynamic capabilities of the plate can also reduce stress on fixation screws or pins and bone into which the plate is fixed.

### BRIEF DESCRIPTION OF THE DRAWINGS

FIGS. 1 and 2 illustrate front views of variations of the plate.

FIG. 3 illustrates a back view of a variation of the plate.

FIGS. 4 through 6 illustrate front views of variations of the plate.

FIGS. 7 and 8 are perspective and front views, respectively of a variation of the plate.

FIG. 9 illustrates that variation of the plate of FIGS. 7 and 8 in a longitudinally extended configuration.

FIGS. 10 through 14b are front views of variations of the plate.

FIGS. 15, 16, 17 and 18 are perspective, side, front, and top views, respectively of a variation of the plate.

FIGS. 19 and 20 illustrate perspective and front views, respectively of two variations of the plate side-by-side.

FIGS. 21 and 22 are front views of longitudinally expanded and contracted configurations, respectively, or a variation of the plate.

FIGS. 23 and 24 illustrate front views of variations of the plate.

FIGS. 25 and 26 are perspective and front views, respectively, of a variation of the plate.

FIGS. 27 and 28 are front and perspective views, respectively, of a variation of the plate.

FIG. 29 is a perspective view of a variation of the plate separate from three variations of cams.

FIG. 30 is a perspective view of a variation of the plate with two variations of cams inserted into the plate and separate from three variations of cams.

FIGS. 31, 32 and 33 are front, side and top views of a method of changing the configuration of the plate. Variations of three separate cams are also shown in FIG. 31.

FIG. 34 is a method of changing the configuration of the plate. Variations of three separate cams are also shown.

FIGS. 35 and 36 are perspective and top partially see-through views, respectively, of a variation of the plate.

FIGS. 37 and 38 are cross-sectional views Y-Y and Z-Z, respectively, of the plate of FIGS. 35 and 36.

FIGS. 39a and 40 are front and perspective views, respectively, of a variation of the plate in a longitudinally expanded configuration.

FIGS. 39b, 39c and 39d are front views of variations of the plate of FIG. 39a.

FIGS. 41 and 42 are front and perspective views, respectively of the plate of FIGS. 39a and 40 in a longitudinally contracted configuration.

FIGS. 43 and 44 are perspective and front views, respectively, of a variation of a plate in a disassembled configuration.

FIGS. 45 and 46 are perspective and front views, respectively, of a variation of the plate of FIGS. 43 and 44 in an assembled, longitudinally expanded configuration.

FIGS. 47 and 48 are perspective and front views, respectively, of a variation of the plate of FIGS. 43 and 44 in an assembled, longitudinally contracted configuration.

FIGS. 49 and 50 are front and perspective views, respectively, of a variation of the plate in a longitudinally expanded configuration.

FIGS. 51 and 52 are front and perspective views, respectively, of the plate of FIGS. 49 and 50 in a longitudinally contracted configuration.

FIGS. 53 through 57 illustrate side views of various embodiments of the plate.

FIGS. 58 and 59 illustrate perspective views of various embodiments of the plate.

FIGS. 60 and 61 illustrate side views of an embodiment of a deployment tool in first and second configurations, respectively.

FIG. 62 illustrates a side cutaway view of an embodiment of a deployment tool.

FIGS. 63 and 64 illustrate side cutaway views of an embodiment of a deployment tool and a front view of an embodiment of the plate in first and second configurations, respectively.

FIGS. 65 and 66 illustrate front views of various embodiments of the plate.

FIGS. 67 and 68 illustrate a perspective view of various embodiments of the plate

FIGS. 69 through 71 illustrate front views of various embodiments of the plate.

FIGS. 72 and 73 illustrate perspective views of embodiments of the plate.

FIGS. 74 and 75 are front views of variations or methods for longitudinally contracting variations of the plate.

FIGS. 76 through 78 are front views of a variation of a method for longitudinally contracting a variation of the plate.

FIG. 79 illustrates cross-section A-A of FIG. 76.

FIG. 80 illustrates cross-section B-B of FIG. 77.

FIG. 81 illustrates a variation of a torquing tool adjusting the cam in a cross-sectional view similar to A-A.

FIG. 82 illustrates top, side and front views of a variation of the cam.

FIGS. 83 through 85 illustrate a variation of a method of attaching and detaching the torquing tool to the cam.

FIG. 86 is a front view of a variation of the cam.

FIGS. 87 through 90 illustrate close-up views of the area proximal to the cam saddle on the plate.

FIGS. 91 and 92 illustrate lateral views of a spinal column.

FIGS. 93 and 94 illustrate anterior views of a spinal column.

FIGS. 95 and 96 illustrate anterior views of a spinal column with an embodiment of the plate attached thereto in first and second configurations, respectively.

FIG. 97 through 99 illustrate lateral views of a method of mounting various embodiments of the plate with mounting screws to the spinal column.

FIGS. 100 through 101 illustrate lateral views of a spinal column with an embodiment of the plate and a mounting tool.

FIG. 102 illustrates a lateral view of a spinal column with an embodiment of the plate mounted thereto.

FIGS. 103 and 104 illustrate anterior views of a spinal column with an embodiment of the plate attached thereto.

FIGS. 105 and 106 are front and side views of a variation of the plate.

FIGS. 107, 108 and 109 are front, side and front views, respectively illustrating methods for altering the dimensions of a variation of the plate.

FIGS. 110 and 111 illustrate a method for longitudinal contraction of the plate when attached to the spine.

FIGS. 112 and 113 illustrate a method for angular shift of the plate in the coronal or frontal plane when the plate is attached to the spine.

FIGS. 114 and 115 illustrate a method for angular shift of the plate in the sagittal plane when the plate is attached to the spine.

### DETAILED DESCRIPTION

FIG. 1 illustrates an adjustable plate for spinal stabilization, fusion, and decompression. The plate 1 can be deformed or otherwise transformed by stretching, expanding or compressing. The plate 1 can be resiliently or deformably transformed, for example by lengthening, shortening, curving or twisting. The plate 1 can be transformed, for example, by a tool that can engage the plate and compress, expand, torque, or otherwise twist the plate, or combinations thereof. The plate 1 can be transformed to adjust the mounting holes to align with the target attachment sites on the bones or soft tissue of the patient. The plate 1 can be fixedly attached (e.g., at the mounting holes) to the target attachment sites on the bones. The plate 1 can be deformed or otherwise transformed before, during, or after the plate 1 is delivered to the target site, or combinations thereof. The plate 1 can be transformed in situ. For example, the plate 1 can be expanded or lengthened to create a higher distraction force between adjacent vertebrae. The plate 1 can have a curved configuration, for example, to match the anatomy of the patient. The plate 1 can be deformed or otherwise transformed to adjust the mounting hole positions to match the desired attachment sites on the vertebra.

The plate 1 can deform after the plate is attached to the target bone. For example, natural stresses from the patient's biomechanics can cause the plate to absorb stress and transform (e.g., deformably or resiliently).

The plate 1 can be configured to mate to any polyaxial screw systems (e.g., from Alphatec Spine, Inc., Carlsbad, Calif., Stryker Corp. of Kalamazoo, Mich.; Biomet, Inc. of Warsaw, Ind.; DePuy, Inc. of Warsaw, Ind.; Medtronic, Inc. of Minneapolis, Minn., or combinations thereof). The plate can be deformed once, twice, or more times.

FIG. 1 illustrates that the plate 1 can have one or more plate deformation apertures or cells 8. The cells 8 can facilitate and/or direct deformation of the plate. The cells 8 can be configured as circles, ellipses, ovals, triangles, squares, rectangles, pentagons, hexagons, other polygons, or combinations thereof. The plate can have, for example, from about one cell to about 100 cells, more narrowly from about four cells to about 10 cells, for example about seven cells.

The plate 1 can have one or more struts 6. The struts 6 can define the boundaries of the cells 8. The plate 1 can have a plate deformation area 5. The plate deformation area 5 can have the struts 6 and the cells 8. The struts 6 can be configured to have a cross section that is uniform, tapering, buttressed, reinforced, solid, hollow, or combinations thereof.

The plate 1 can have one, two or more end flanges 3. The end flanges 3 can be at the ends of the plate 1, for example bordering or otherwise surrounding the deformation area 5.

The plate 1 can have a number of attachment or screw holes 2, or sockets therethrough. The screw holes 2 can be threaded, for example, for receiving and attaching to one or more bone screws, pins, brads, other fixation elements, or combinations thereof. The plate 1 can have from about two screw holes 2 to about 15 screw holes 2, more narrowly from about four screw holes 2 to about eight screw holes 2, for example about four screw holes 2. The screw holes 2 can be located on the end flanges 3 of the plate 1. The plate 1 can have one, two, three, four or more screw holes 2 located, for example, on the end flanges 3 of the plate 1.

The plate 1 can have one, two, or more plate deformation adapters 4. The plate deformation adapters 4 can be sockets, holes, flanges 3, or combinations thereof. The plate deformation adapters 4 can be configured to receive and engage a plate deformation tool. The plate deformation too can deform the plate 1 from a first configuration (e.g., longitudinally contracted or longitudinally expanded) to a second configuration (e.g., the opposite of the first configuration) and/or vice versa. The plate 1 can have from about two plate deformation adapters 4 to about 15 plate deformation adapters 4, more narrowly from about three plate deformation adapters 4 to about eight plate deformation adapters 4, for example about four plate deformation adapters 4.

The plate 1 can have about four holes for plate deformation adapters 4. The plate deformation adapters 4 can be a fixation point through which force can be applied using the plate deformation tool. The plate deformation adapters 4 can be arranged around the perimeter of the plate 1, toward the longitudinal ends of the plate 1, on the end flanges 3, on the deformation area 5, in the middle of the plate 1, or combinations thereof.

The plate 1 can have a pre-deformation width B and a pre-deformation height C. The pre-deformation width B can be from about 0.318 cm (0.125 in.) to about 10 cm (4 in.), for example about 3.8 cm (1.5 in). The pre-deformation height C can be from about 2.5 cm (1 in.) to about 10 cm (4 in.), for example about 5 cm (2 in.).

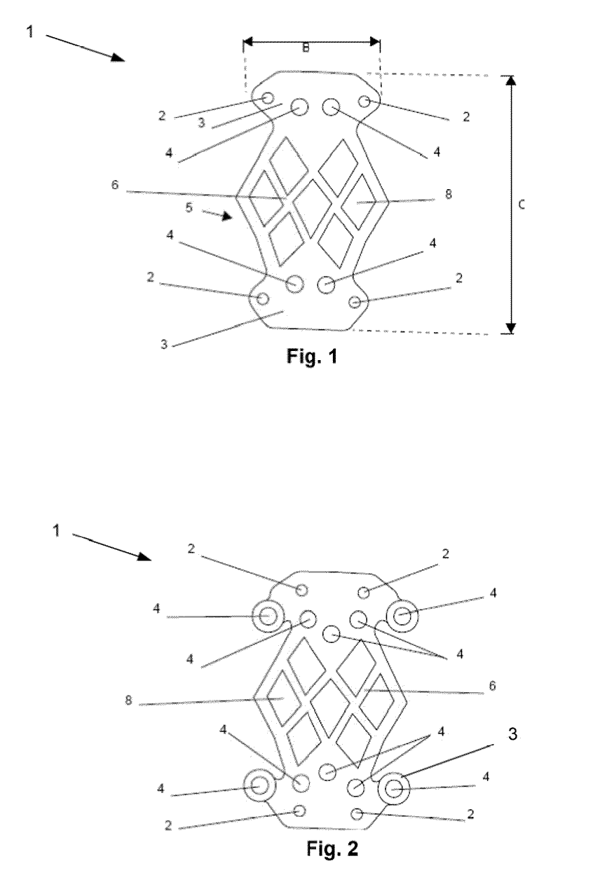

FIG. 2 illustrates that the plate 1 can be provided with 10 plate deformation adapters 4 disposed therethrough. FIGS. 2 and 4 further illustrate that the plate deformation adapters 4 can be provided in leverage enhancing extended locations, such as on ears of the plate or flanges 3. FIG. 2 illustrates that the flanges 3 can extend laterally from the longitudinal ends of the plate 1. FIG. 4 illustrates that the flanges 3 can extend laterally from around the longitudinal median of the plate 1.

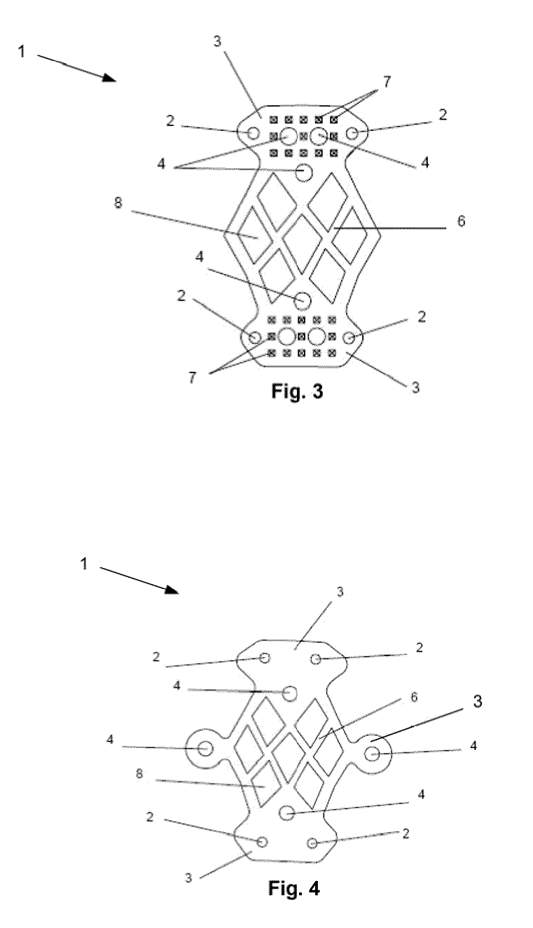

FIG. 3 illustrates that the surface of the vertebral attachment area of the plate 1 can be textured, for example with knurling or a coating, or smooth, or a combination thereof. The plate 1 can have a texture, for example, to enhance the security of the connection between the plate 1 and the vertebra to which the plate is attached, to encourage bony healing, growth, or fusion, or combinations thereof. The plate 1 can be coated with a bone growth factor, such as a calcium base or materials listed herein. The texture can be rough texturing 7, small bumps, spikes, anchors, brads, hooks, or combinations thereof.

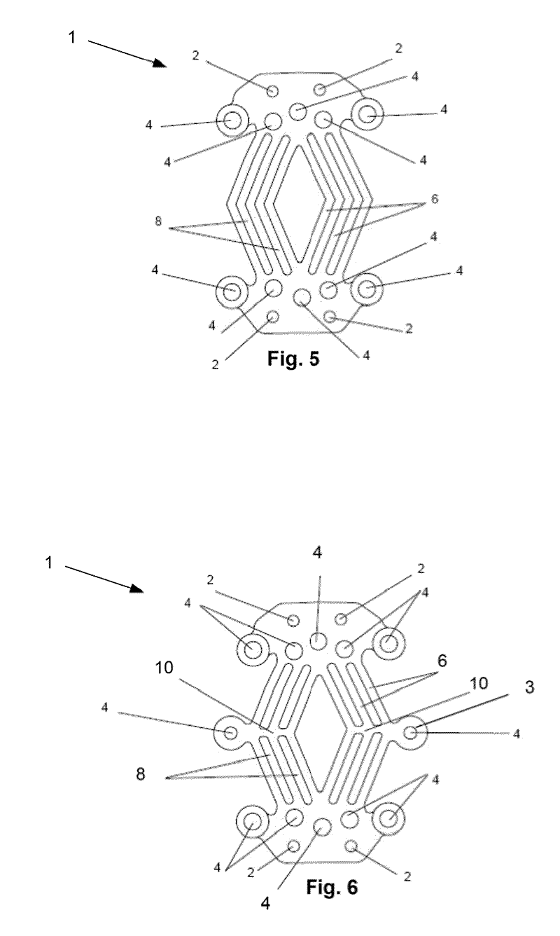

FIG. 5 illustrates that a set of one or more struts 6 can span the longitudinal length of the plate deformation area 5. Cross-struts 10 can be absent in the transverse direction (e.g., connecting the longitudinally-oriented struts to each other). The struts 6 can have a configuration having one or more substantial angles (as shown) or lengths with a substantial radius of curvature.

FIG. 6 illustrates that one or two cross-struts 10 can extend laterally or transversely, for example, at about the middle of the longitudinal length of the plate 1. The cross-struts 10 can have one, two or more flanges 3 that can extend from the sides of the plate 1. The flanges 3 can have plate deformation adapters 4.

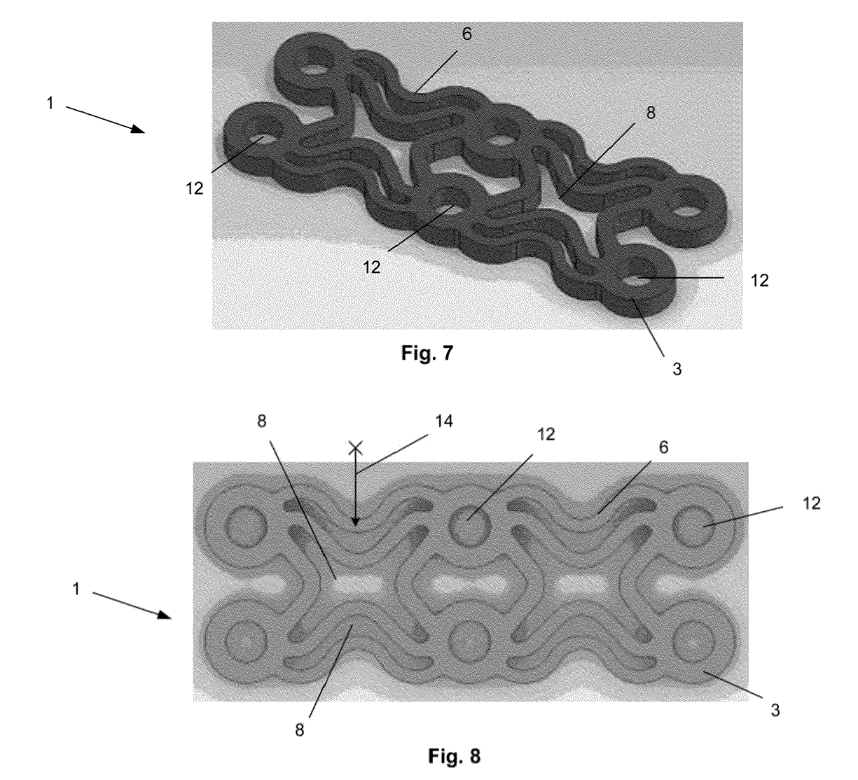

FIGS. 7 and 8 illustrate that some or all of the attachment holes 12 can be in flanges 3 that extend longitudinally from one or both longitudinal ends of the plate 1. The struts 6 can have one or more strut radius of curvature 14. The struts 6 can be grouped into proximally adjacent sets throughout the plate 1. For example, sets of two struts 6 each can extend in the longitudinal direction from each of the longitudinally distal attachment holes 12. Also for example, a set of single, double or more struts 6 can connect the flange 3 surrounding an attachment hole 12 to a laterally adjacent flange 3 surrounding an attachment hole 12.

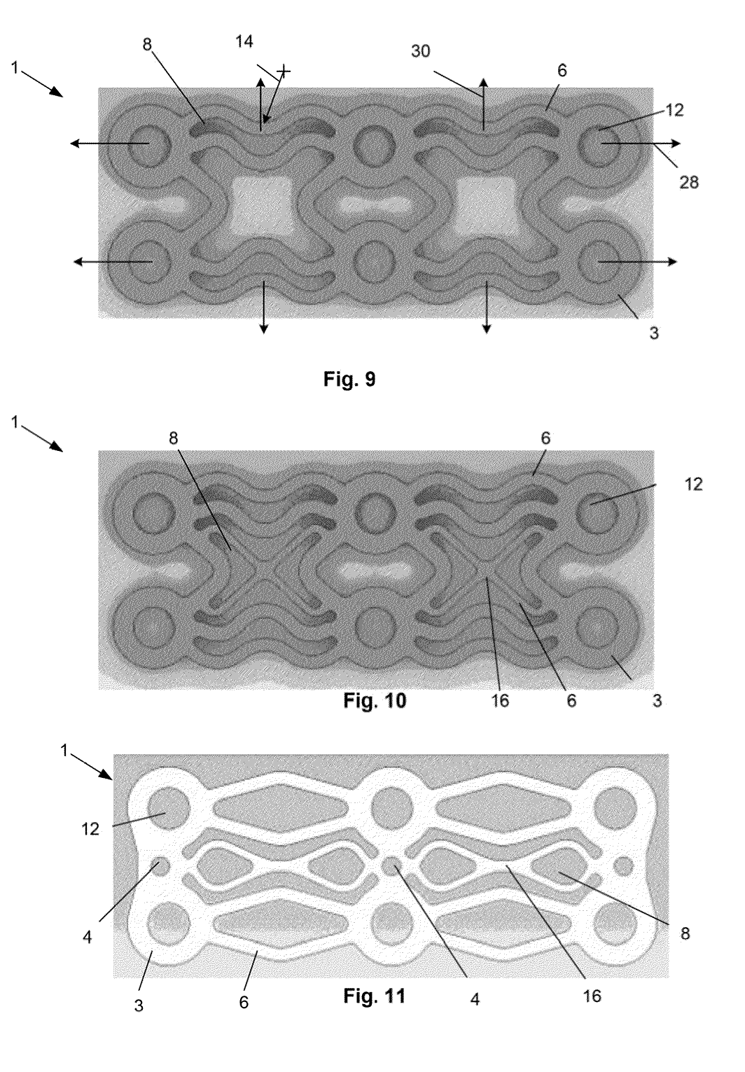

FIG. 9 illustrates the plate of FIGS. 7 and 8 in a longitudinally extended configuration. A force, as shown by arrows 28, can be applied at any of the attachment holes 12, for example longitudinally extending (e.g., tensile force), compressing or torquing/twisting all or a portion of the length or side of the plate 1 without extending, compressing or torquing/twisting (or performing the opposite translation) the remainder of the plate 1. The resulting deformation, shown by arrows 30, of the struts 6 from the applied force(s) can radially extend, as shown in FIG. 9, or contract the struts 6. For example, the strut radius of curvature 14 can decrease, as shown, or increase.

FIG. 10 illustrates that the struts 6 can be integral or attached to each other at one or more joints 16. The struts 6 can rotate at or adjacent to the joints 16.

FIG. 11 illustrates that the plate 1 can have deformation adapters between laterally and/or longitudinally adjacent attachment holes 12.

Holes labeled as plate deformation adapters or attachment holes herein can be used for either or both of attachment of a deployment or deformation tool or a fixation device (e.g., screw).

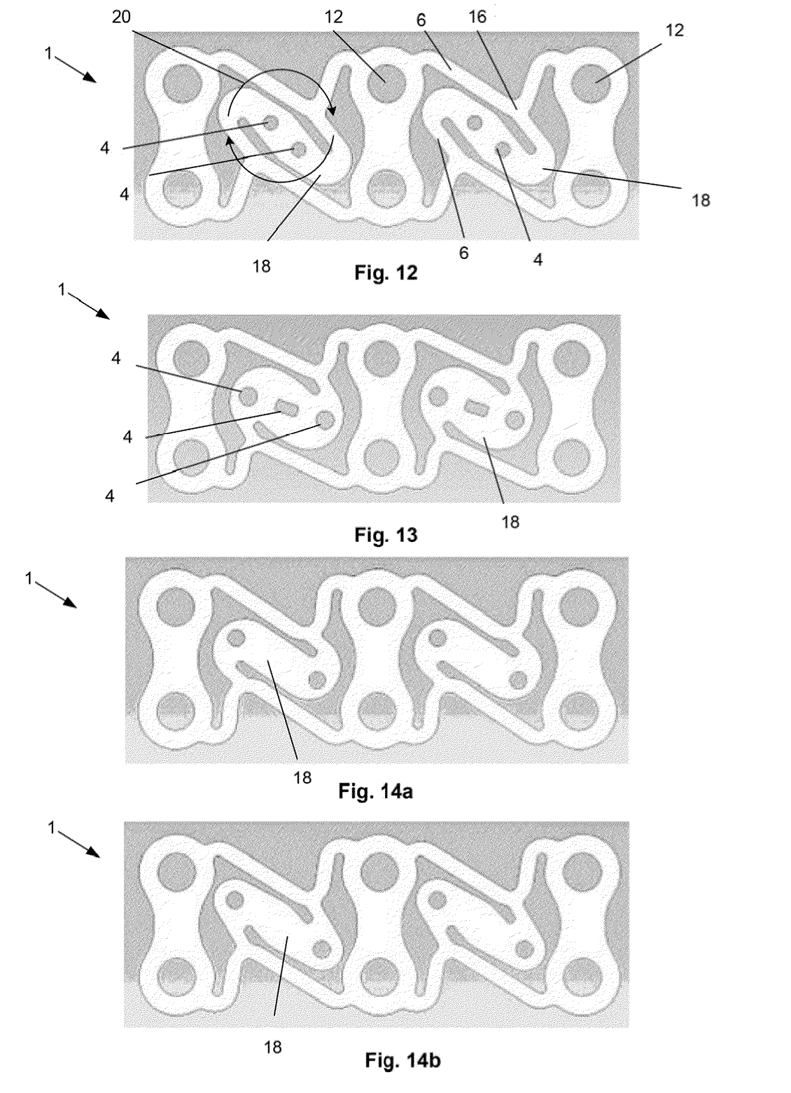

FIG. 12 illustrates that the plate 1 can have one, two or more controlled deformation panels 18. The panels can be attached to or integral with two or more struts 6. The controlled deformation panels 18 can be rotated in either direction. For example, a rotational tool can be attached to the plate deformation adapters 4. The tool can then rotate the controlled deformation panel 18. When the controlled deformation panel 18 is rotated as shown by arrows 20, the controlled deformation panel 18 can deformably or resiliently transform the surrounding struts 6, thereby extending the length of the plate 1. When the controlled deformation panels 18 are rotated opposite as shown by the arrows, the plate 1 can be longitudinally shortened.

FIG. 13 illustrates that the controlled deformation plate 18 can have three plate deformation adapters 4. One or more of the controlled deformation adapters 18 can be differently configured from the other deformation adapters 4. For example, the middle plate deformation adapter 4 can be rectangular. The remaining plate deformation adapters 4 can be circular.

FIG. 14a illustrates that the controlled deformation panel 18 can be rounded and elongated. FIG. 14b illustrates that the controlled deformation panel 18 can be diamond-shaped.

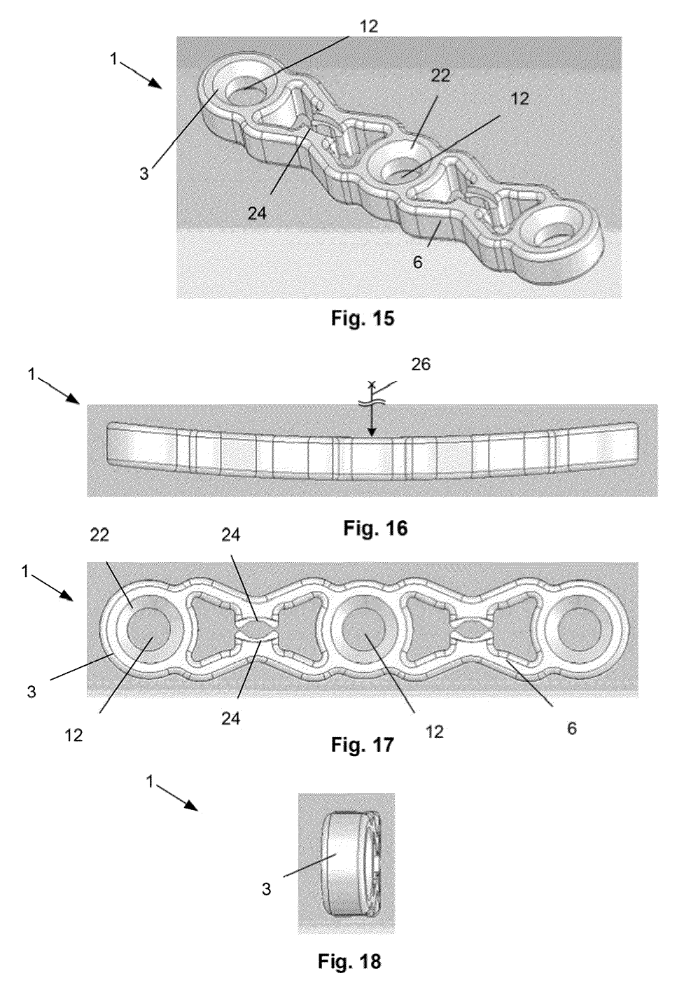

FIGS. 15 through 18 illustrate that the plate can have recessed (as shown) or raised) attachment hole seats 22 surrounding the attachment holes 12. The attachment hole seats 22 can be beveled and/or chamfered. The plate 1 can have internal stops 24. Each stop 24 can be adjacent to another internal stop 24. The stops 24 can extend laterally inward from the struts 6. The stops 24 can have a flat or curved (as shown) surface facing internally to the plate 1. During longitudinal contraction of the plate 1, for example during deployment and/or during use from regular biomechanics of the patient, each stop 24 can abut an adjacent stop 24 to substantially stop longitudinal contraction of the plate 1.

The plate can be used for dynamic stabilization of any portion of the spine, such as the lumbar, cervical, thoracic, sacral, or combinations thereof.

A deployment tool (e.g., plate deformation tool) can be attached between adjacent stops 24. The deployment tool can press laterally against the stops 24 to force the stops 24 laterally outward resulting in longitudinal expansion of the plate 1.

The plate can have a plate radius of curvature 26. The plate radius of curvature 26 can approximate the radius of curvature of the spine length where the plate 1 is deployed, for example the cervical, lumbar or thoracic spine curvature, or combinations thereof. The plate radius of curvature 26 can be with respect to an axis offset and parallel to the transverse axis of the plate 1, as shown, and/or to an axis offset and parallel to the longitudinal axis,

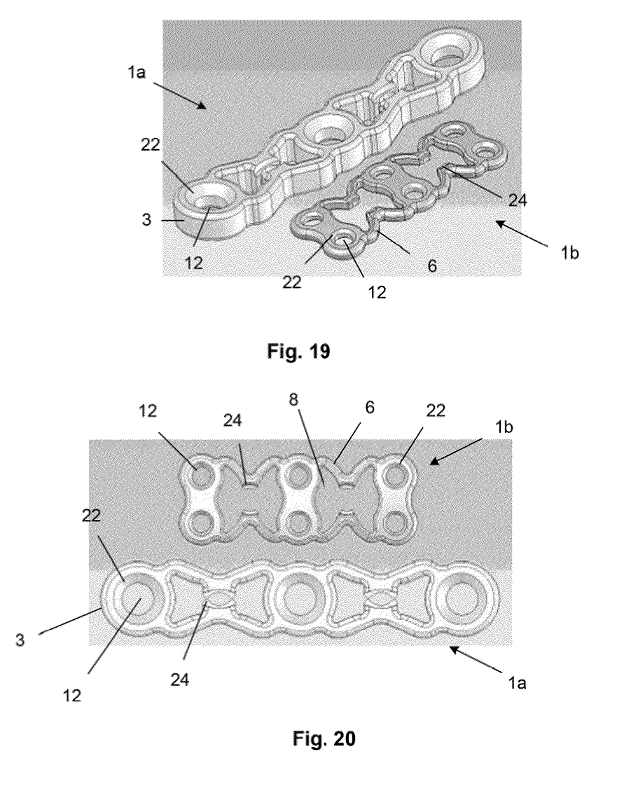

FIGS. 19 and 20 illustrate a first plate 1a (e.g., of FIGS. 16 through 19) and a second plate 1b. The second plate 1b can have a different configuration than the first plate 1a. The second plate 1b can have two times as many attachment holes 12. For example, a first attachment hole 12 can be laterally symmetric with a second attachment hole 12.

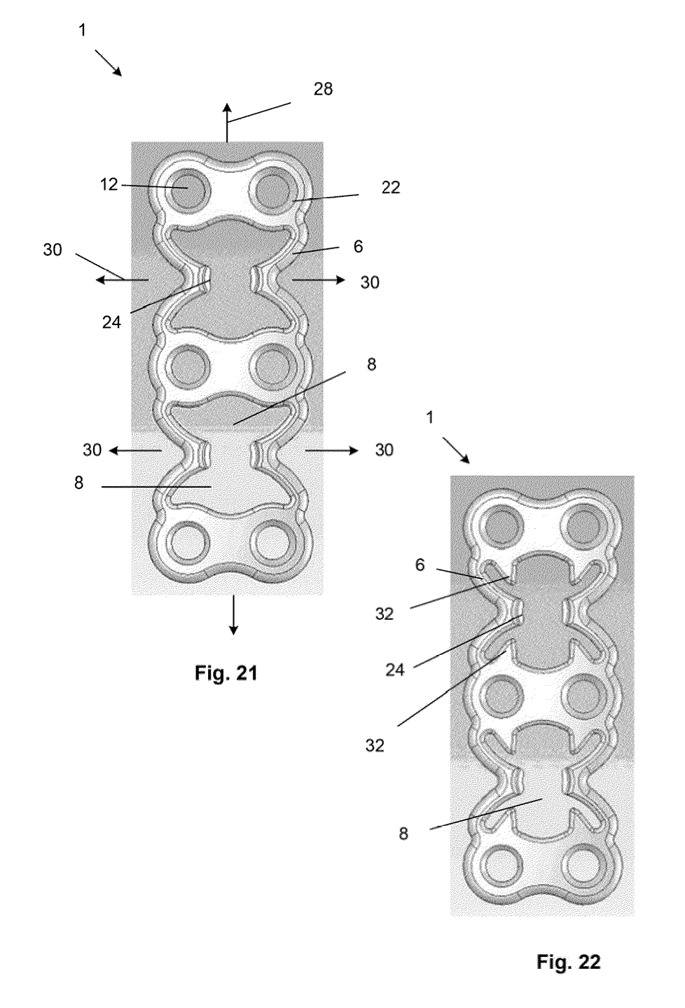

FIG. 21 illustrates that the applied tensile force, as shown by arrows 28, (e.g., via any combination of the attachment holes 12) can force the struts 6 to deform laterally outwardly, as shown by arrows 30.

FIG. 22 illustrates that the plate 1 can have length abutments 32 adjacent to the internal side of each strut 6. When the plate 1 or a portion of the plate 1 is longitudinally contracted, the length abutments 32 can form an interference fit with (i.e., abut) the struts 6. The abutment of the struts 6 against the length abutments 32 can set the minimum longitudinal length for each section of the plate 1.

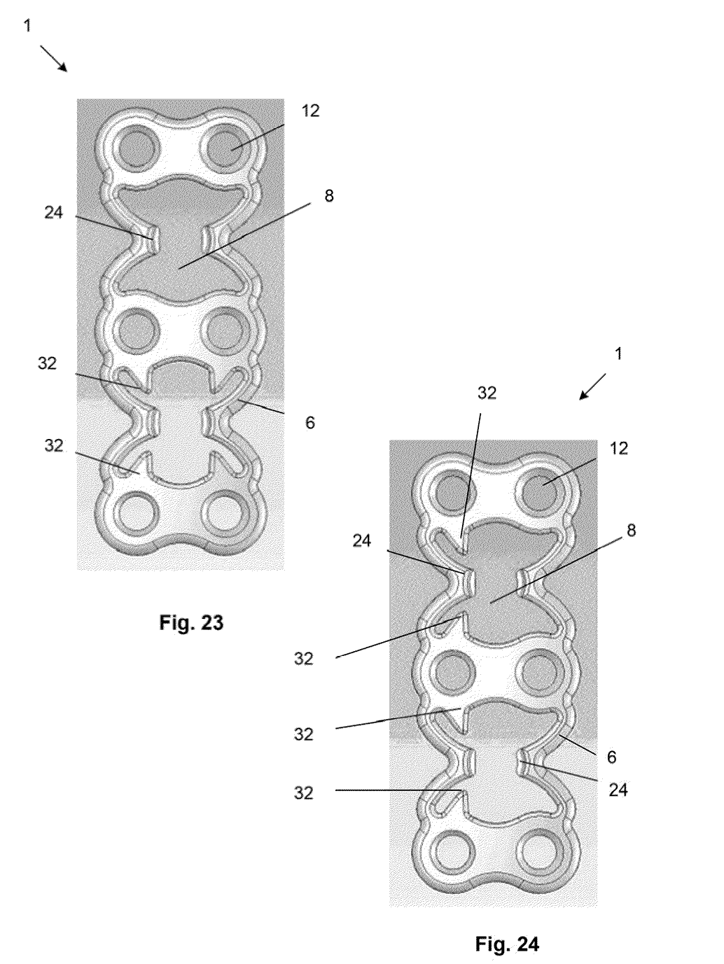

FIG. 23 illustrates that the length abutments 32 can be on one or more longitudinal sections of the plate 1 and absent from the remaining longitudinal sections of the plate 1. For example, the length abutments 32 can be on one longitudinal half 34 of the plate 1, as shown.

FIG. 24 illustrates that the length abutments 32 can be on one or more lateral sections of the plate 1 and absent from the remaining lateral sections of the plate 1. For example, the length abutments 32 can be on one lateral half of the plate 36, as shown.

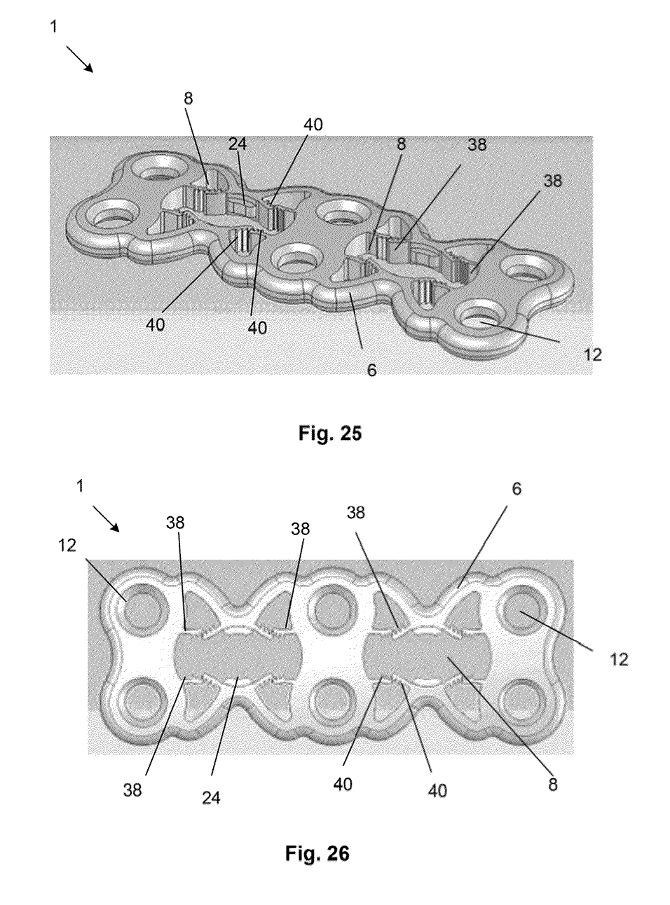

FIGS. 25 and 26 illustrate that the plate 1 can have locking arms 38. Pairs of adjacent locking arms 38 can be configured to have unidirectional or bidirectional slidable interface with each other (e.g., against opposing faces of the locking arms 38). Pairs of adjacent locking arms 38 can be configured to form releasable interference fits with each other. For example, the locking arms 38 can have unidirectional or bidirectional teeth 40 facing an adjacent opposing locking arm 38. The teeth 40 can be configured to latch or ratchet to teeth 40 on an opposing locking arm 38. The locking arms 38 can have teeth 40, brads, textured surfaces, hook and loop surfaces, single or multiple latches, or combinations thereof. The locking arms 38 can extend from the struts 6, joints 16, flanges 3 surrounding the attachment holes 12 or otherwise, or combinations thereof.

The locking arms 38 can fixedly attach to each other and adjustably lock to fit the minimum length of the plate 1.

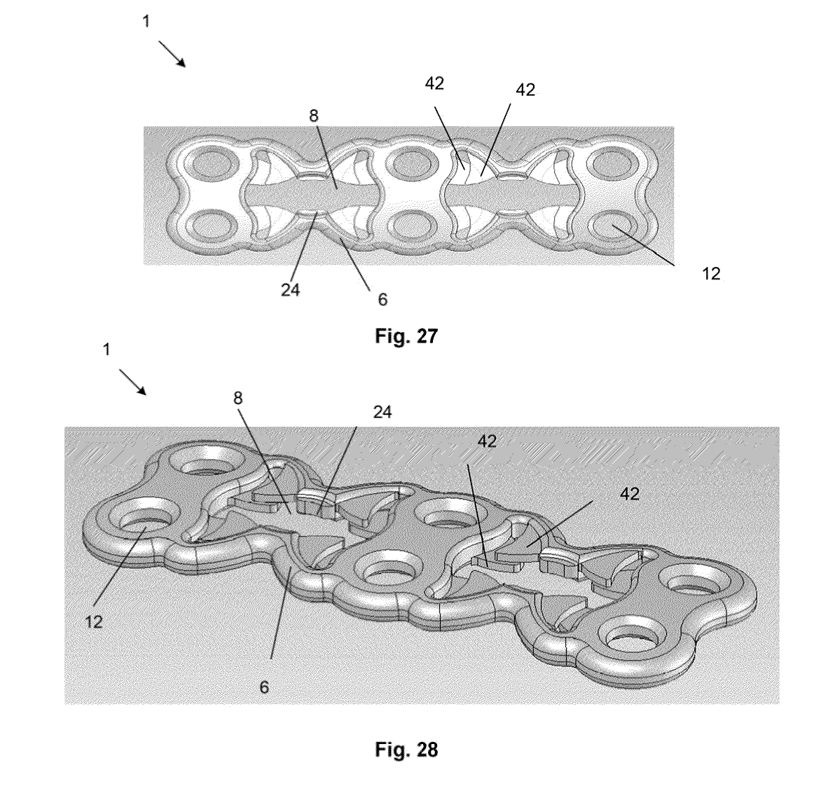

FIGS. 27 and 28 illustrate that the plate 1 can have abutment panels 42. The abutment panels 42 can extend from the struts 6, joints 16, flanges 3 surrounding the attachment holes 12 or otherwise, or combinations thereof. The abutment panels 42 can interference fit against other abutment panels 42, struts 6, joints 16, flanges 3 surrounding the attachment holes 12 or otherwise, or combinations thereof. The abutment panels can provide a hard stop to prevent overexpansion and/or overcontraction of the plate.

Pairs of adjacent abutment panels 42 can be configured to have unidirectional or bidirectional slidable interface with each other (e.g., against opposing faces of the locking arms 38). Pairs of adjacent abutment panels 42 can be configured to form releasable interference fits with each other. For example, the abutment panels 42 can have unidirectional or bidirectional teeth 40 facing an adjacent opposing abutment panel 42. The teeth 40 can be configured to latch or ratchet to teeth 40 on an opposing abutment panel 42. The abutment panels 42 can have teeth 40, brads, textured surfaces, hook and loop surfaces, single or multiple latches, or combinations thereof.

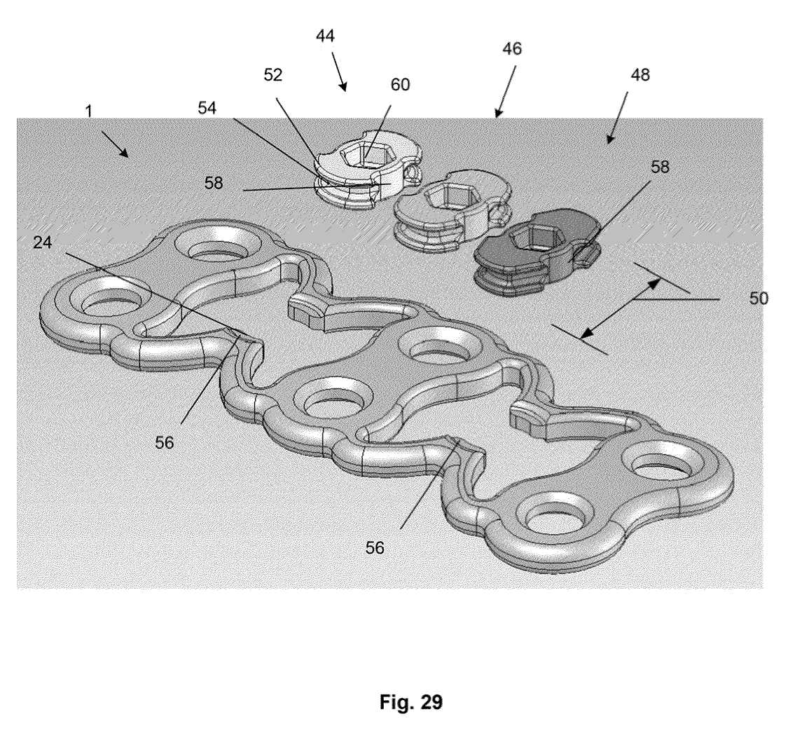

FIG. 29 illustrates that the plate 1 can be used in conjunction with one or more cams. One or more first cams 44, second cams 46, and/or third cams 48 can be separate from the plate 1. The first 44, second 46, and third cams 48 can have progressively longer cam lengths 50. The cam size (e.g., first, second or third) can be selected based on the desired configuration of the implanted and deployed plate.

The cams can have cam lips 52 surrounding a cam groove 54. The plates 1 can have cam lip receivers 56 on either side of the stop 24. The stop 24 can be configured to slidably attach in the cam groove 54. The cam lips 52 can be configured to slidably attach to the cam lip receivers 56. The stop 24 can be curved to seat the cam groove 54.

The cam can have a cam side slot 58. The cam side slot 58 can be absent of cam lips 52.

The cam can have one or more cam attachment ports 60. The cam attachment ports 60 can be configured to receive a tool for transmitting a translational force or torque. The cam attachment port 60 can be a hex port (as shown), a flat, Phillips, or spanner-head screw driver slot, or combinations thereof.

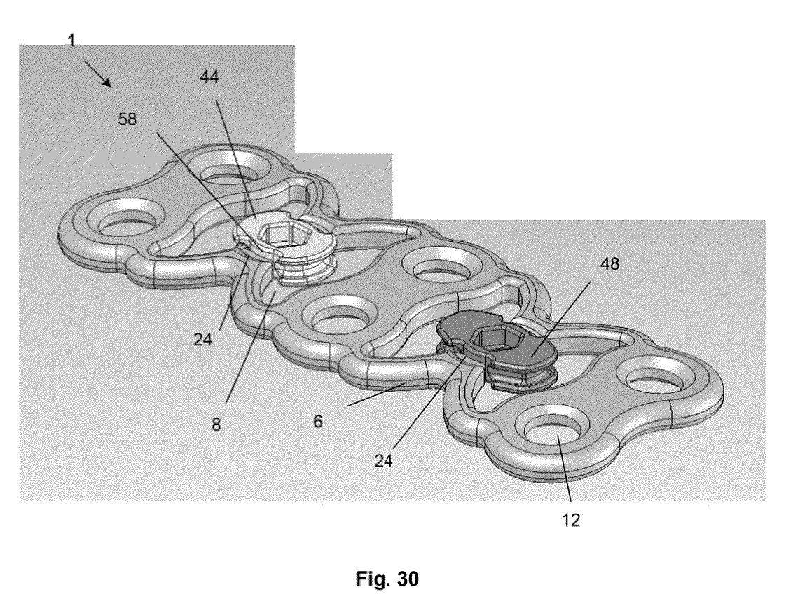

FIG. 30 illustrates that cams can inserted between laterally adjacent stops 24 on the plate 1. The same or different (as shown) sized cams can be used for a single plate 1. The longitudinal axis of the cam can be aligned with the longitudinal axis of the plate 1. The cams can be pushed between the slots so the cam side slots 58 arte frictionally fit against the stops 24.

FIG. 31 illustrates that the cams can be rotated, as shown by arrows 62, to laterally force out the stops 24 and struts 6, longitudinally extending the plate 1, as shown by arrows 64. One or both cams can be rotated. Either or both cams can be rotated so the longitudinal axis of the cam is substantially perpendicular to the longitudinal axis of the plate 1. The cam lips 52 can fit around the stops 24. The cams can be rotated to lock the plate open, and/or longitudinally extend the plate, and/or to unlock the plate, and/or to longitudinally shorten the plate.

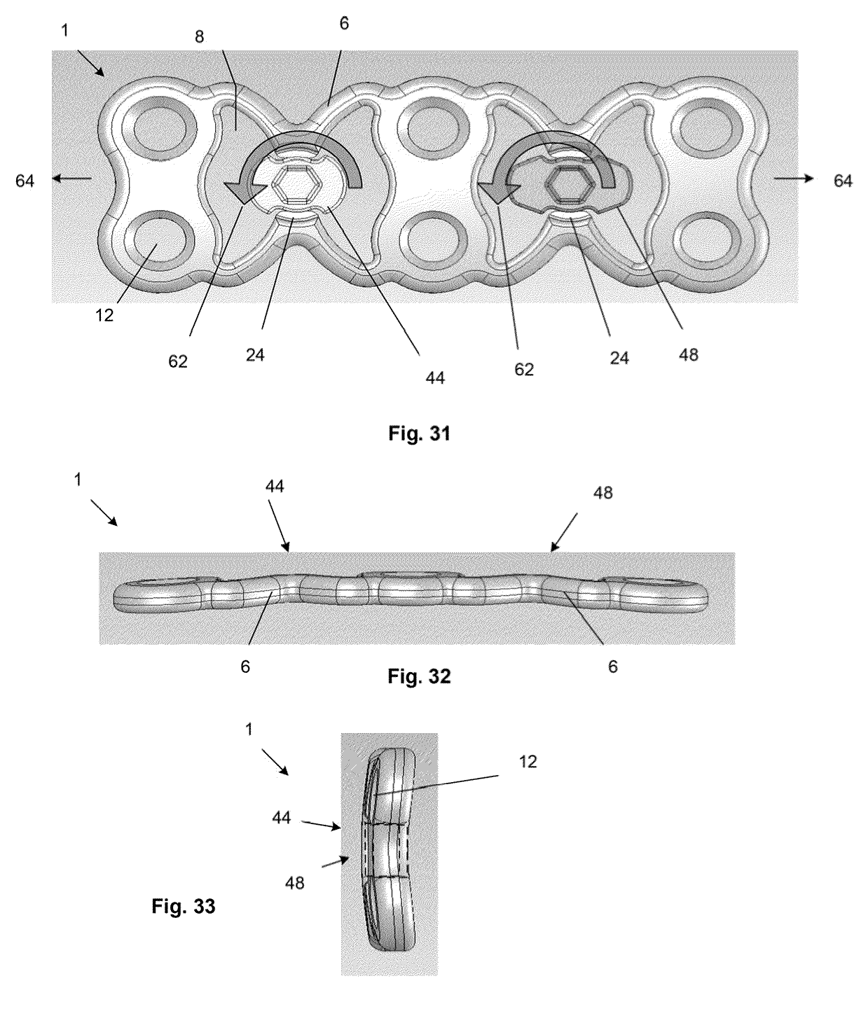

FIGS. 32 and 33 illustrate that the profile of the cams can be lower than the profile of the plate 1, for example so the cams do not extend in front of or behind the plate 1, as shown. The cams can be configured to bear biomechanical loads, for example, in any direction.

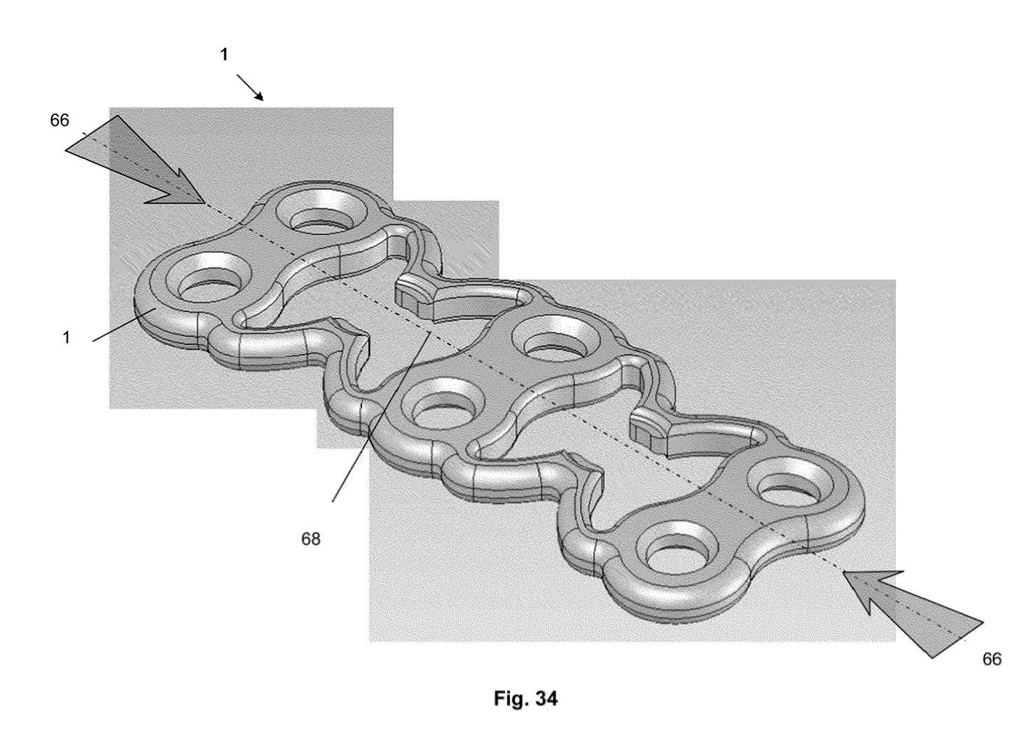

FIG. 34 illustrates that a longitudinal force, as shown by arrows 66, can be applied to the plate 1. The plate 1 can be longitudinally compressed or contracted along the longitudinal axis 68 when one or both cams are not inserted into the plate 1.

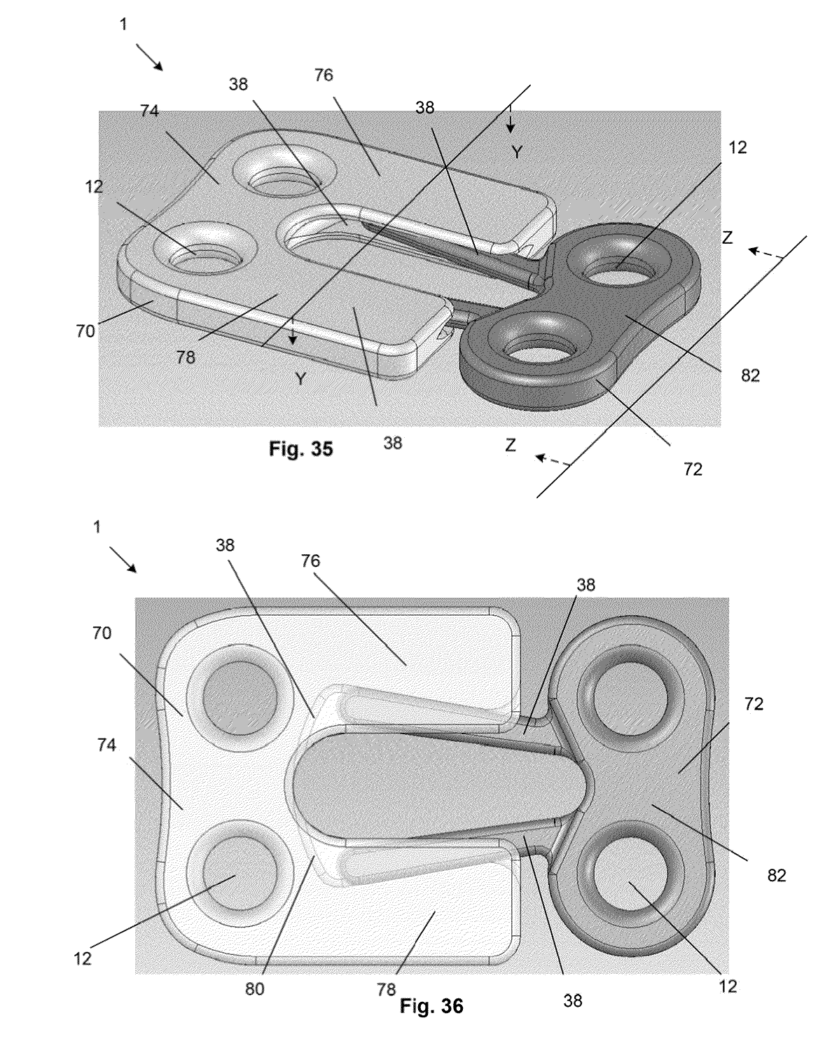

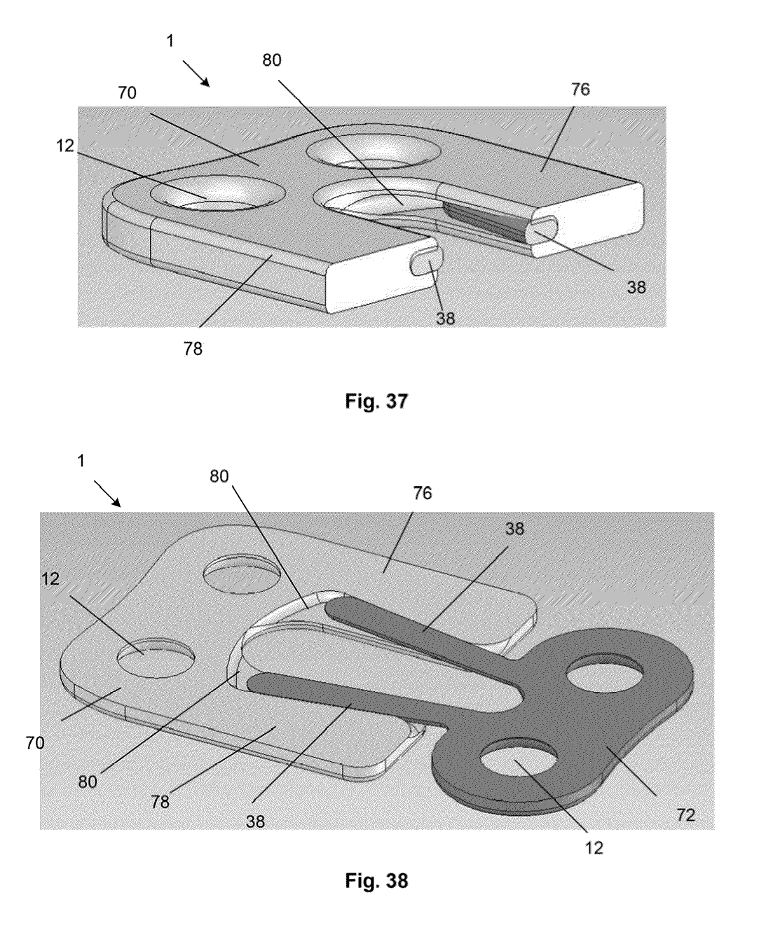

FIGS. 35 through 38 illustrates that the plate 1 can have a plate first section 70 and a plate second section 72. The plate first section 70 can have a plate first section head 74 that can have one or more attachment holes 12. One, two or more legs (e.g., the first leg 76 and the second leg 78) can extend from the plate first section head 74. The legs can have longitudinally disposed arm slots 80. The arm slots 80 can open to the radial inside of the legs. The arm slots 80 can expand radially relative to the longitudinal axis 68 of the plate 1 as the arms slots 80 approach the plate first section head 74.

The plate second section 72 can have a plate second section head 82. The plate second section head 82 can have one, two or more attachment holes 12. One, two or more locking arms 38 can extend from the plate second section head 82. The locking arms 38 can extend from the second section head at an angle so the locking arms 38 extend radially outward relative to the longitudinal axis 68 of the plate 1 as the locking arms 38 extend away from the plate second section head 82. The locking arms 38 can be resilient or deformable. The locking arms 38 can be placed into the arm slots 80, for example by resilient or deformably bending the arms to clear the legs and enter the arm slots 80. Deformable arms can be deformed radially outward after placement into the arm slots 80.

The plate 1 can be adjusted before during or after implantation, for example by the surgeon or by the application of natural biomechanical forces during use. The plate second section 72 can be moved closer to or further away from the plate first section 70.

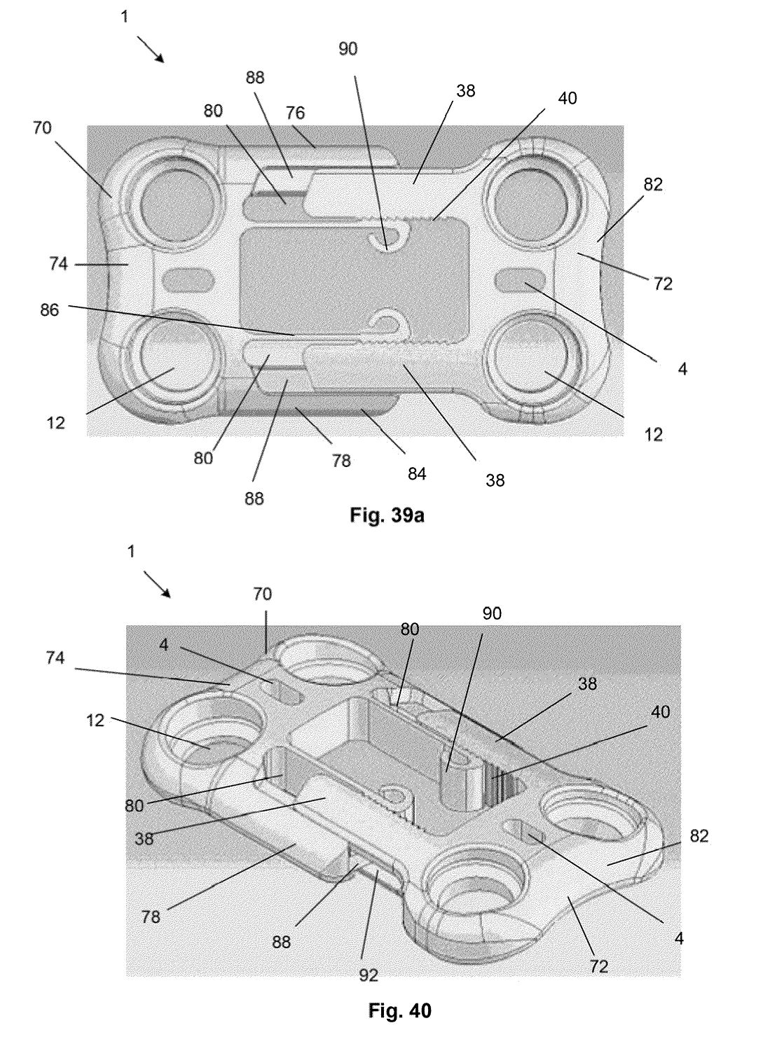

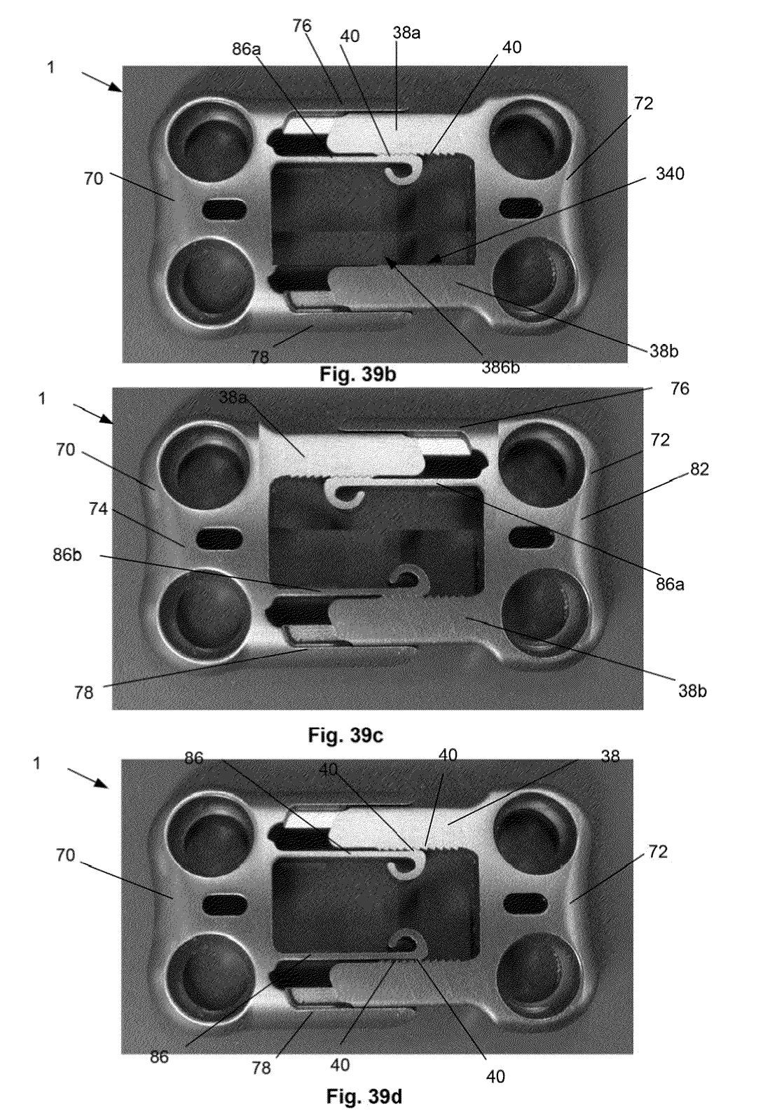

FIGS. 39a and 40 illustrate that the plate first section head 74 and/or the plate second section head 82 can have plate deformation adapters 4 that can be configured to interface or otherwise removably attach from one or more deployment tools. Bone screws 188, or other permanent or temporary devices to attach the respective plate section to bone or other tissue, can be removably or permanently inserted through the plate deformation adapters 4, attachment holes 12, arms slots 80, the space between the locking arms 38, the space between the flexible legs 86, or combinations thereof, for example to insert into bone or other tissue. The plate first section 70 can be slidably attached to the plate second section 72.

The arms and legs can be integral with the plate section heads at first ends and have terminal ends at second ends opposite to the first ends.

The legs can have arm slots 80 configured to unidirectionally or bidirectionally slidably receive one or more locking arms 38. The locking arms 38 can be substantially longitudinally slidable in the arm slots 80 longitudinally with respect to the plate 1, and/or the locking arms 38 can be slid in the arm slots 80 at a substantially non-zero angle with respect to the longitudinal axis of the plate 1.

The arm slots 80 can be defined between a rigid leg section 84 and a flexible leg section 86. The flexible leg section 86 can be resilient or deformable. The legs, for example in the rigid 84 or flexible leg section 86, can have rails 88 configured to be slidably received by the locking arms 38 and/or align each locking arm 38 with the respective leg. The legs can have unidirectionally or bidirectionally engaging teeth 40 on an inner surface of the arm slots 80, for example on the rigid 84 or flexible leg section 86. The unidirectional teeth can allow sliding in a first direction (e.g., when the plate first section 70 and the plate second section 72 are brought toward one another), and interference fit in a second direction (e.g., when the plate first section 70 and the plate second section 72 are moved away from one another) when to prevent the locking arm 38 from being withdrawn from the arm slot 80. Each leg can have one or more arm handle release handles 90, for example on the rigid 84 or flexible leg section 86.

The locking arms 38 can extend parallel to the longitudinal axis 68 of the plate 1 from the second section head 82. The locking arms 38 can have unidirectional or bidirectional teeth 40. The teeth 40 on the locking arm 38 can be configured to unidirectionally or bidirectionally interference fit the teeth 40 on the arm slot 80. The locking arms 38 can have grooves 92 configured to slidably receive the rails 88 on the legs.

The arm release handle 90 can be resiliently or deformably pulled or otherwise bent or rotated to release the teeth 40. For example, the teeth 40 on the radial inside of the locking arm can disengage from the teeth 40 on the radial outside of the flexible leg section 86. For example, when the teeth 40 are configured to unidirectionally interference fit opposed teeth 40, the flexible leg section 86 can be pulled or otherwise rotated away from the locking arm 38 to enable movement of the locking arm (and the plate second section 72) in the direction otherwise opposed by the engaged teeth 40.

A locking aim 38 can have a first flexible leg section 86 on the radial inside of the locking arm 38, as shown in FIGS. 39a, and 40-42, and a second flexible leg section 86 on the radial outside of the locking arm 38. The teeth 40 on the radial outside of the first flexible leg section 86 can unidirectionally interference fit the teeth 40 on the radial inside of the locking arm 38 in a first direction. The teeth 40 on the radial inside of the second flexible leg section 86 can unidirectionally interference fit the teeth 40 on the radial outside of the locking arm 38 in a second direction, for example equal to or opposite the first direction. For example, if the first direction is equal to the second direction, the first and the second flexible leg sections 86 can be bent or otherwise rotated away from the locking arm 38 to enable the locking arm 38 to slide in the first or second direction. If the first direction is opposite to the second direction, the first flexible leg section 86 can be bent or otherwise rotated away from the locking arm 38 to enable the locking arm 38 to slide in the first direction, and the second flexible leg section 86 can be bent or otherwise rotated away from the locking arm 38 to enable the locking arm 38 to slide in the second direction. Therefore, the motion of the plate second section 72 with respect to the plate first section 70 can be controlled and/or prevented in either direction along the arm slot based on the rotation of the flexible leg sections 86, for example as rotated at the target site or outside of the patient's body by a deployment or control tool.

The teeth 40 on any variation herein can be teeth 40, brads, textured surfaces, hook and loop surfaces, snaps, magnets, ridges, single or multiple latches, or combinations thereof.

FIG. 39b illustrates that the second leg 78 can have a rigid leg section 84 on the radial outside (as shown) or inside of the second locking arm 38b. The second leg 78 can have no flexible leg section 86, as indicated by arrow 386b. The second locking arm 38b can be free of teeth 40, as shown by arrow 340. The second locking arm 38b can be free of teeth 40 whether or not the corresponding leg has a flexible leg section. The first leg 76 can have a first flexible leg section 86a. The flexible leg section 86a can have teeth on the radial outer side of the first flexible leg section 86a. The first locking arm 38a can have teeth 40 on the radial outer side of the first locking arm 38a. The teeth 40 on the first locking arm 38a can interface with the teeth 40 on the first flexible leg section 86a.

FIG. 39c illustrates that the second leg 78 and/or the first locking arm 38a can be integral with (as shown), or attached to, and extend from the first plate section head 74. The first leg 76 and/or the second locking arm 38b can be integral with (as shown), or attached to, and extend from the second plate section head 82. The first locking arm 38a can be slidably received by the first leg 76. The second locking arm 38b can be slidably received by the second leg 78.

FIG. 39d illustrates that one or both the flexible leg sections 86 can have no or exactly one tooth 40 on the sides of either or both flexible leg sections 86 that face the locking arms 38. In addition or alternatively, the sides of either or both the locking arms 38 that face the flexible leg sections 86 can have exactly one tooth 40.

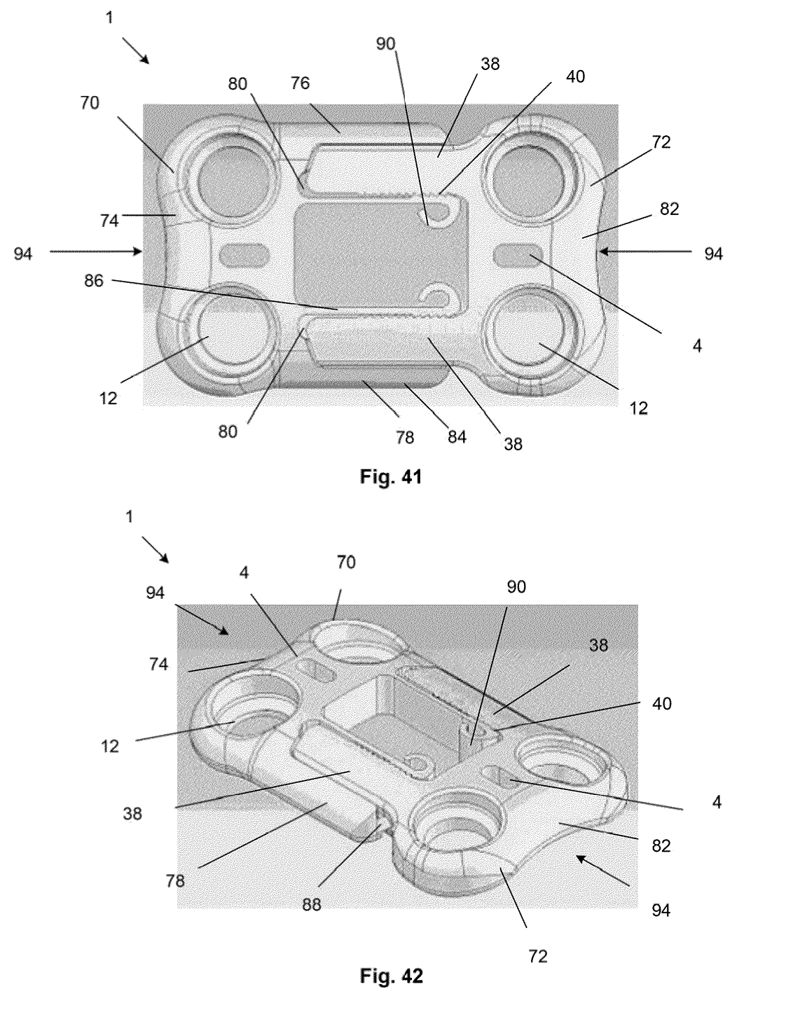

FIGS. 41 and 42 illustrate the a longitudinally compressive force can be applied, as shown by arrows 94, to the plate first section 70 and the plate second section 72, longitudinally contracting the plate 1. The force can be applied through a deployment tool via the plate deformation adapters 4 and/or by natural biomechanics after implantation. For example, the force can be from the movement of the adjacent bones (e.g., vertebrae) or bone sections into which the plate first section 70 and plate second section 72 are attached via attachment screws or posts through the attachment holes 12.

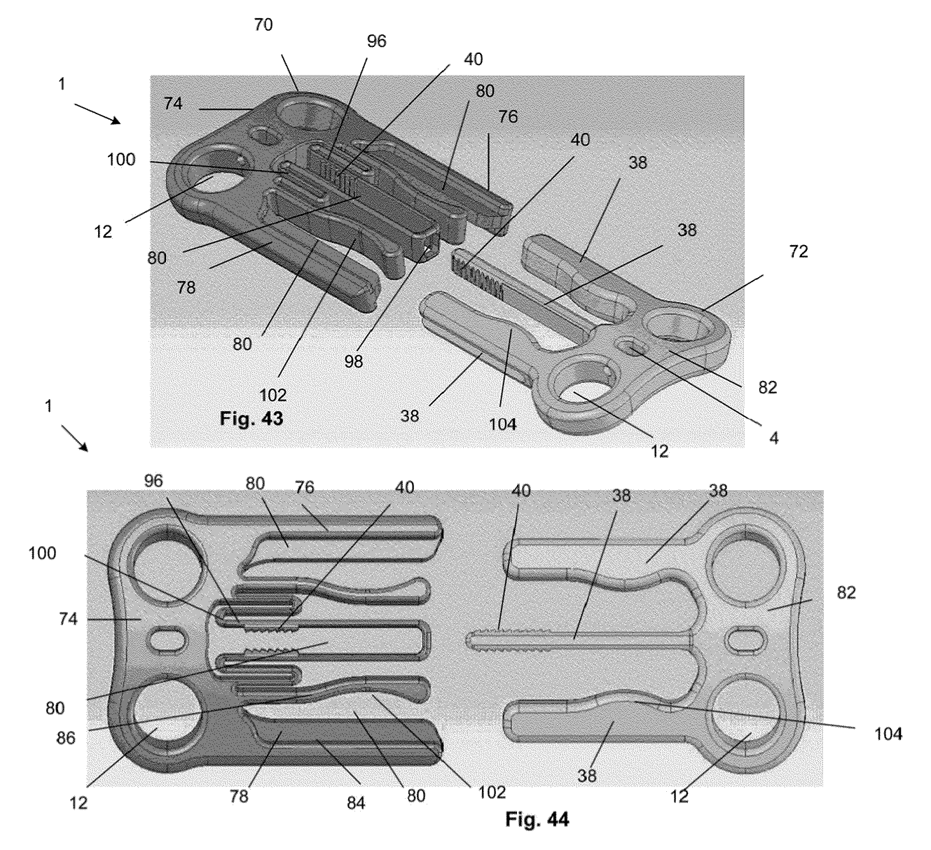

FIGS. 43 and 44 illustrate that the plate first section 70 can be completely separate from the plate second section 72 before or during deployment. The plate first section 70 can have a third leg 96. The third leg 96 can have a third leg port 98. The third leg port 98 can be configured to steer a locking arm 38 into the third leg 96. The third leg 96 can be attached to a resilient leg spring 100. The third leg 96 can have teeth 40 on an internal surface. The first 76 and second legs 78 can have leg divots 102. The leg divots 102 can be configured to removably receive arm bumps 104 on the respective locking arms 38.

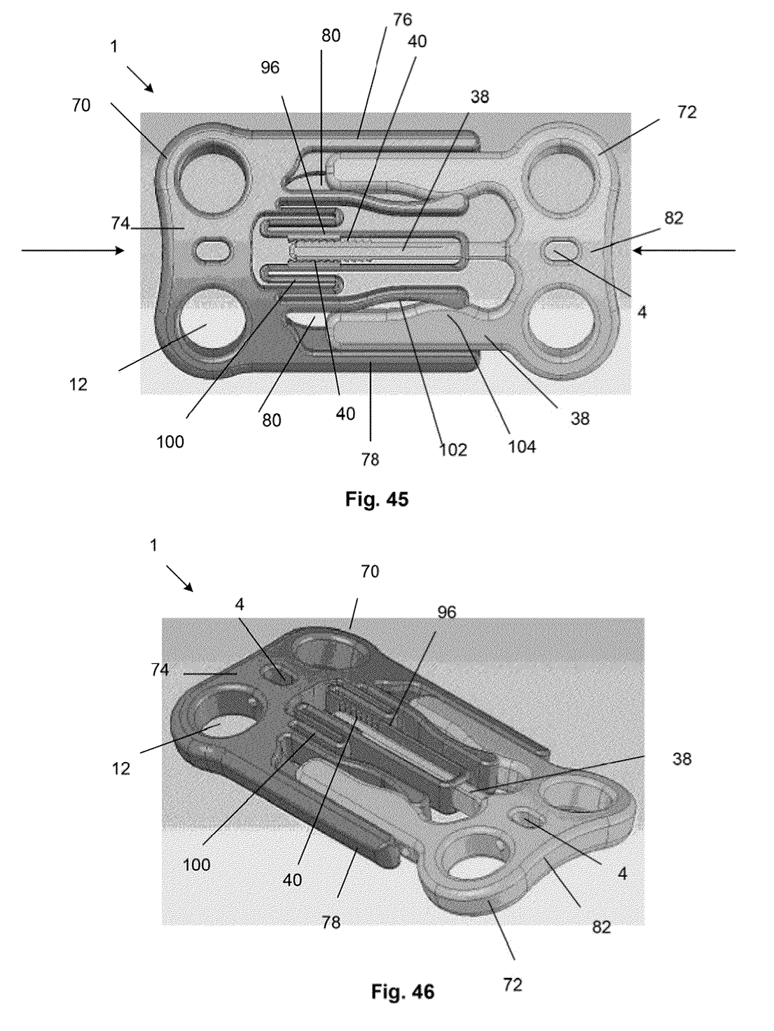

FIGS. 45 and 46 illustrates that the plate first section 70 can slidably receive the plate second section 72. The middle locking arm 38 of the plate second section 72 can enter the third leg port 98. The teeth 40 on the middle locking arm 38 can engage the teeth 40 on the inside of the third leg 96.

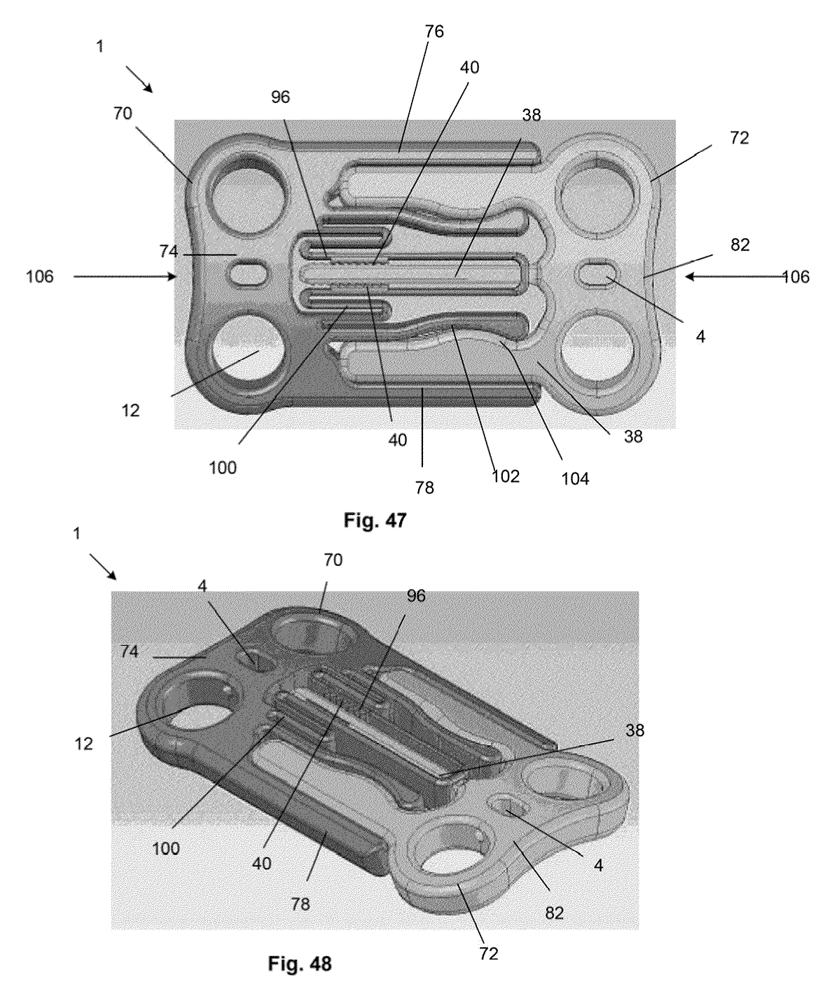

FIGS. 47 and 48 illustrate that a compressive force, as shown by arrows 106 can be applied to contract the plate length. The arm bumps 104 can seat into the leg divots 102.

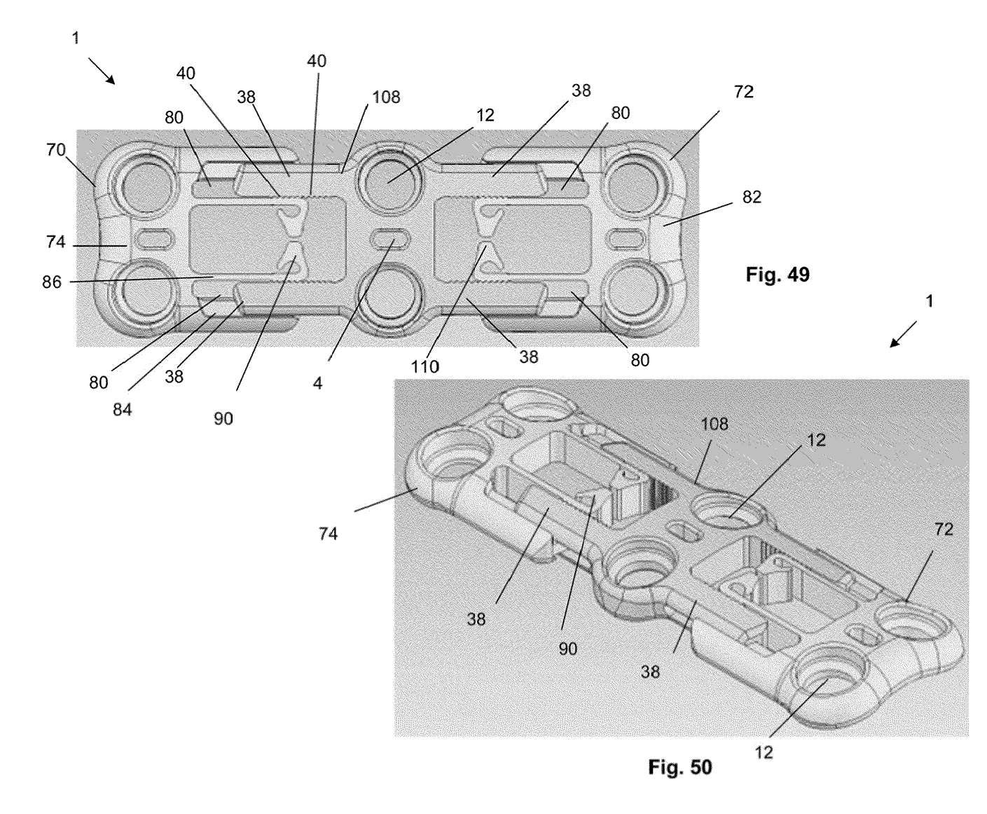

FIGS. 49 and 50 illustrate that the plate 1 can have one or more plate intermediate sections 108 that can be slidably received by other plate intermediate sections 108 and/or the plate first section 70 and plate second section 72. Multiple intermediate plate sections 108 can be used between the plate first section 70 and plate second section 72. For example, the sequence of plate sections from one side to another can be: (1) plate first (end) section, (2) plate first intermediate section, (3) plate second intermediate section, (4) plate third intermediate section, and (5) plate second (end) section. Multiple plate intermediate sections 108 can be configured to be slidably attached to one another. The plate intermediate section 108 can have sets (e.g., one, two or more) locking arms 38 extending in opposite directions. The plate intermediate section 108 can have a plate deformation adapter 4.

The arm release handle 90 can have a bumper 110. The bumper 110 and/or the arm release handle 90 can have a substantially D-shaped configuration, for example an open-D-shaped configuration, as shown, or a closed-D-shaped configuration, or otherwise can have a flat or minimally rounded contact surface pointed toward the laterally opposed arm release handle 90. The arm release handles 90 can be bent or otherwise rotated away from the locking arm 38 to disengage or otherwise release the teeth 40 of the flexible leg section 86 from the teeth 40 of the locking arm 38. When the flexible leg section 86 is rotated away from the locking arm 38 disengage the teeth 40, the locking arm 38 can be translated with respect to the flexible leg section 38. For example, when the locking arms 38 are all disengaged from the locking arms 38, the adjacent plate sections 70 and 72, for example, can be translated away from each other, for example remaining attached or detaching for repositioning of the plate 1 or components or elements thereof or removal of the plate 1 of components or elements thereof. For example, bone screws or other fixation devices can also be removed or can be left in place.

The bumper 110 can be made from the same or a different material than the arm release handle 110. The arm release handle 90 can be configured to interference fit against the laterally opposing arm release handle 90 to limit the range of motion of the arm release handle 90 (e.g., before the arm release handle 90 is bent far enough to plastically deform).

The teeth 40 can be various configurations. For example, the last tooth in the line of teeth 40 can be larger than the remaining teeth 40. The respective last channel between teeth 40 in the opposing teeth 40 can be larger than the remaining channels. The last tooth can then provide extra resistance when the plate 1 is in a fully contracted configuration.

A safety element can be used in the plate 1 to prevent the plate sections from disengaging from each other even if the arm release handle 90 is retracted. The safety element can be a pin that can internally traverse adjacent plate sections. The pin can be placed within elongated channels in the adjacent plate sections. The elongated channels in adjacent plate sections can be configured to overlap when the plate sections are in use.

The safety element can be a significantly larger tooth or teeth 40, as described supra. The safety element can be a latch on the locking arm 38 that intersects and connects to the respective leg during use. A combination of safety elements can be concurrently used.

The intermediate plate section can have one or more legs in lieu of or in addition to the locking arms 38. The plate first section 70 and plate second section 72 can have locking arms 38 and/or legs corresponding to legs and/or locking arms 38, respectively, on the plate intermediate section 108. (Likewise, on the other variations herein, locking arms 38 can be substituted or used in addition to legs, and vice versa, and corresponding legs and/or locking arms 38 can be on the adjacent plate section.)

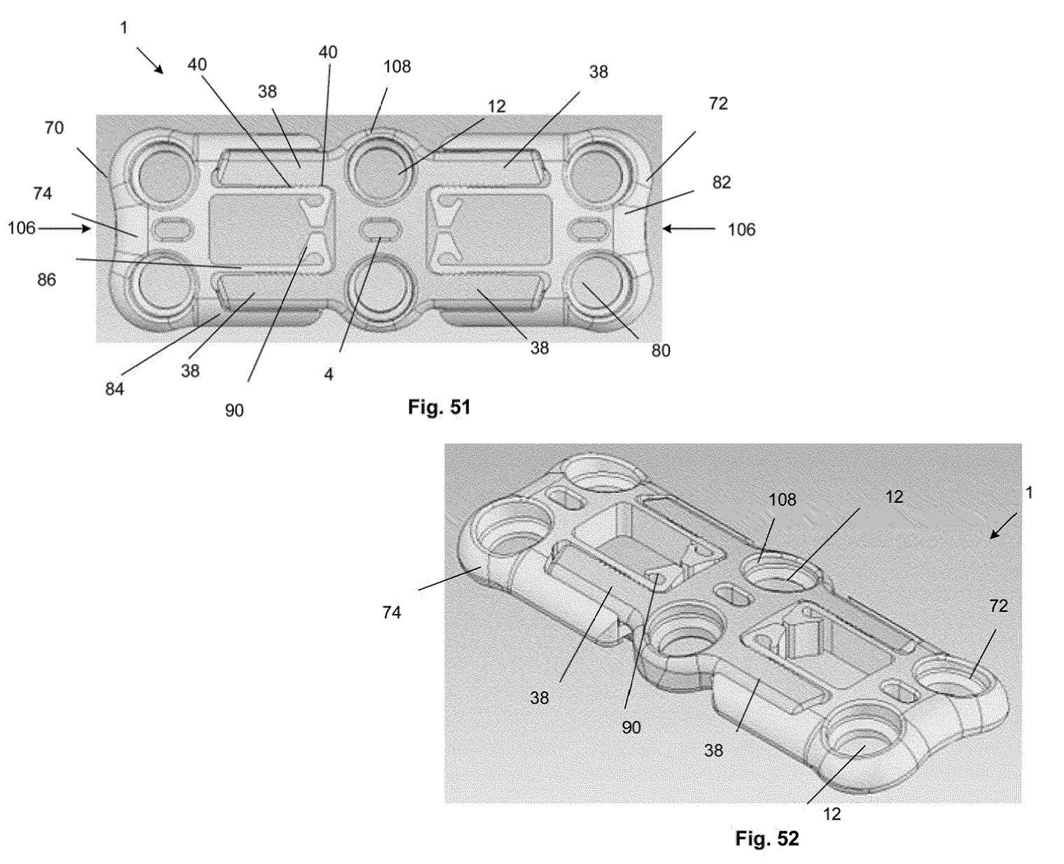

FIGS. 51 and 52 illustrates that a compressive force, shown by arrows 106, can contract the plate length. The compressive force 106 can be deployed on only part (e.g., only between the plate intermediate section 108 and the plate first section 70) or the entire length of the plate, contracting the respective length.

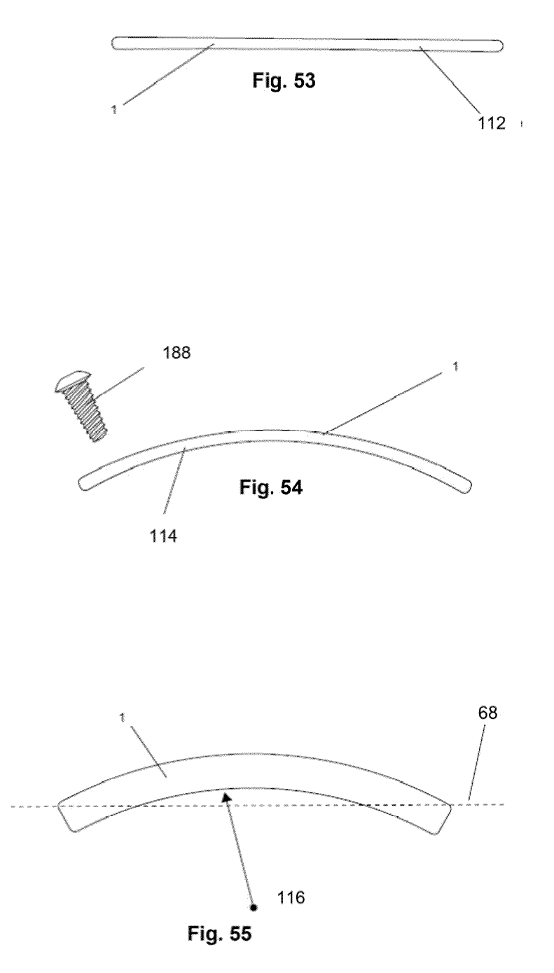

FIGS. 53 through 55 illustrate that the plate 1 can have a uniform thickness. FIG. 53 illustrates that the plate 1 can have a flat profile 112. FIGS. 54 and 55 illustrate that the plate 1 can have a curved profile 114. FIG. 54 illustrates that one or more bone screws 188 can be screwed or slid through the plate 1. The plate 1 can have a flat profile 112 and/or a curved profile 114 with a radius of curvature. The bone screw can be inserted through the curved or flat portion of the plate. FIG. 55 illustrates that the plate 1 can have a radius of curvature 116 along the longitudinal axis 68. The radius of curvature 116 can be from about 10 mm (0.4 in.) to about an infinite radius (i.e., for a flat profile 112), more narrowly from about 250 cm (100 in.), for example about 50 cm (20 in.). The plate can have a flat or curved profile regardless of thickness or thinness, for example, the curved plate can have a thickness of about half the length of the bone screw 188.

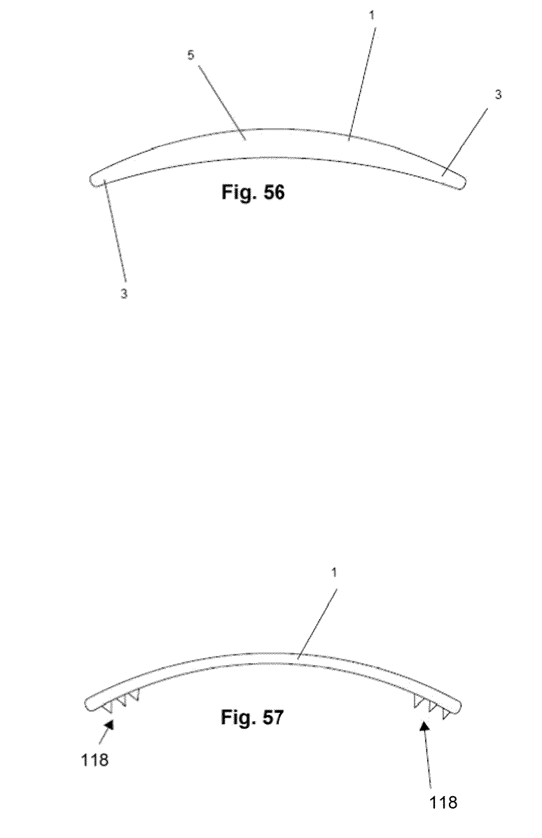

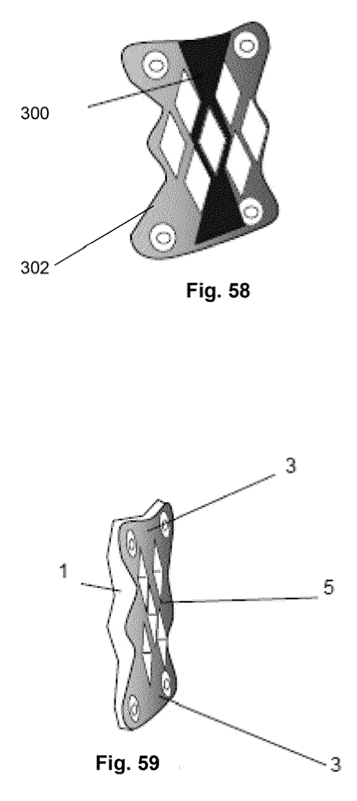

FIG. 56 illustrates that the plate 1 can have a variable thickness. The plate 1 can have a thicker cross-section in the plate deformation area 5 and a thinner cross-section in the end flanges 3. FIG. 58 illustrates that the plate 1 can have thicker cross-sections in some higher strength zones 300 to exhibit stiffer characteristics in these areas, and lower strength zones 302 to exhibit less stiff characteristics.

FIG. 57 also illustrates that the plate 1 can have spikes 118 extending from its surface.

The plate 1 can be transformed using a deployment tool, such as a deformation tool, for example a distraction device. The deployment tool can engage the plate 1, for example with a pair of pointed engagement prongs.

FIGS. 58 and 59 illustrate that the plate can have high strength zones 300 and/or low-strength zones 302. For example, the high-strength zones 300 can be in and around the longitudinal center of the plate, and the low-strength zones 302 can be in and around the plate distal to the central longitudinal axis. The high-strength zones 300 can be configured to resist bending and/or torsion (e.g., have a higher bending modulus than the low-strength zones). The high-strength zones 300 can have thicker cross-sections, and/or different (e.g., stronger) materials, than the low-strength zones 302.

The cells can be circular, oval, square, rectangular, triangular, hexagonal, diamond-shape (as shown), or combinations thereof.

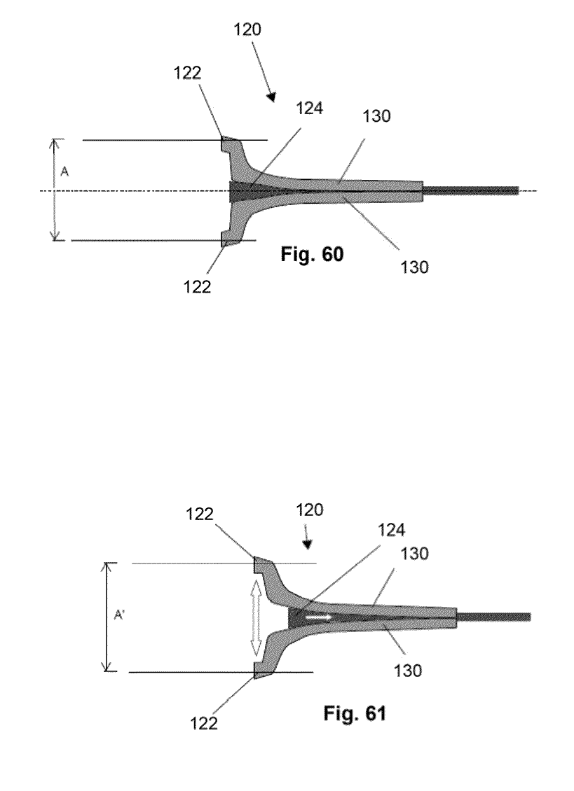

FIGS. 60 and 61 illustrate an embodiment of a deformation tool 120 that has two engagement prongs 122. The deformation tool 120 can be configured to allow the user to insert the engagement prongs 122 into the plate deformation adapters 4 and then apply force to the engagement prongs 122 with respect to one another in order to deform the plate in a desired manner. The deformation tool 120 can have an expander element 124, for example forcibly slidable within an expansion slot by means of a screw rod.

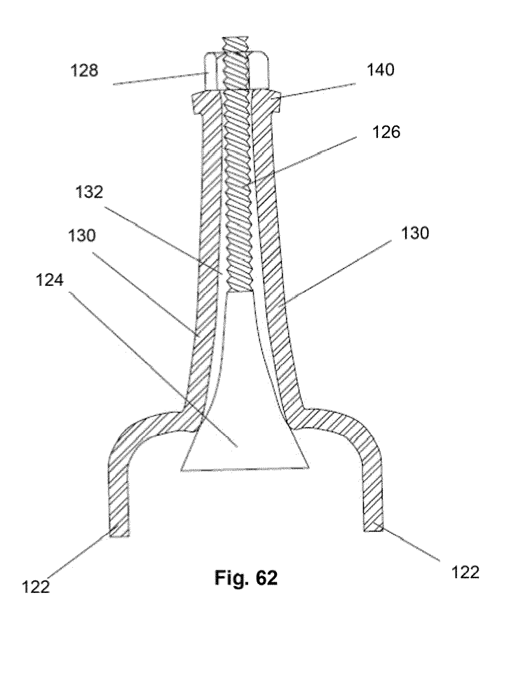

FIG. 61 illustrates a deformation tool that has an expander element 124 forcibly slidable within an expansion slot. FIG. 62 illustrates that the deformation tool 120 can have a screw rod 126 adapted to act upon the expander element 124 and a nut 128 engaged thereto.

FIGS. 60, 61, and 62 illustrate that a deformation tool 120 can have preloaded elastic arms 130, adapted to apply a compressive squeezing force when the expander element 124 is slid toward the engagement prongs 122 in the expansion slot 132, thereby allowing the preloaded elastic arms 130 to squeeze together. The deployment tool 120 can apply either a compressive, squeezing force between the engagement prongs 122 or a tensile, stretching force between the engagement prongs 122, or both.

FIGS. 60 and 61 illustrates that the deformation tool 120 can have a compressed engagement prong 122 spacing A and an expanded engagement prong 122 spacing A′, respectively.

The deformation tool 120 can also applied a twisting force. Two or more deformation tools 120 can also be used in combination to apply a bending force, for example across the deformation area of the plate 1 in order to modify its profile. The engagement prongs 122 may be used to engage any of the various plate deformation adapters 4. For example, the engagement prongs 122 can engage plate deformation adapters 4 directly symmetrically opposed from one another, or on opposite corners of the plate 1, or a combination thereof. The engagement prongs 122 can also be inserted into the screw-holding sockets.

The deformation struts 6 can be configured to affect a particular mode of the deformation in response to an applied to force of a particular direction. For example, the deformation struts 6 can be configured to cause the plate 1 to deform in a direction perpendicular to the direction of the applied force. For example the deformation struts 6 can be configured to react to a lateral compression force by affecting a longitudinal expansion.

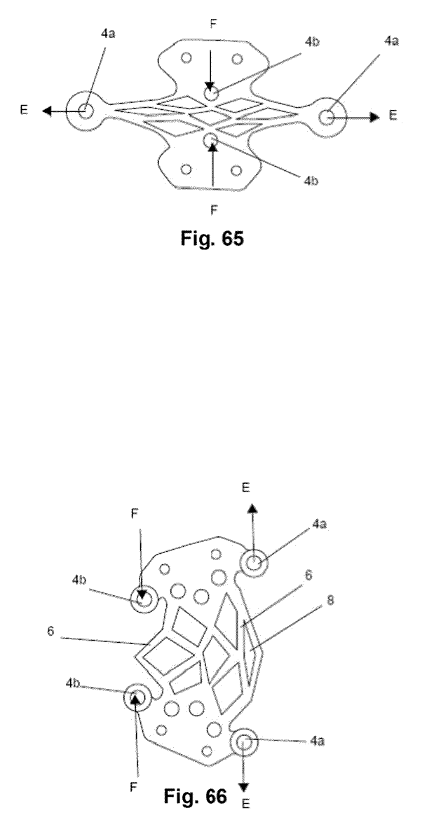

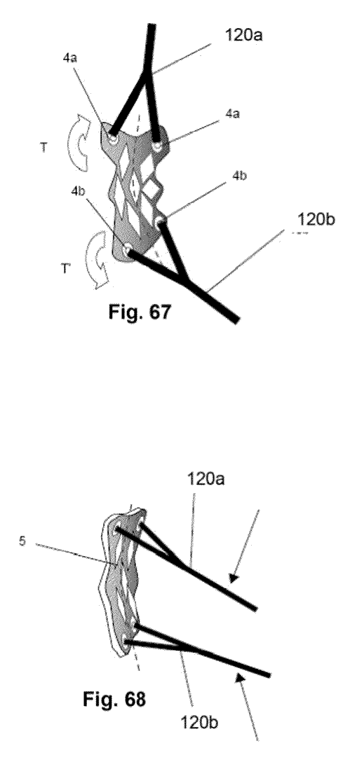

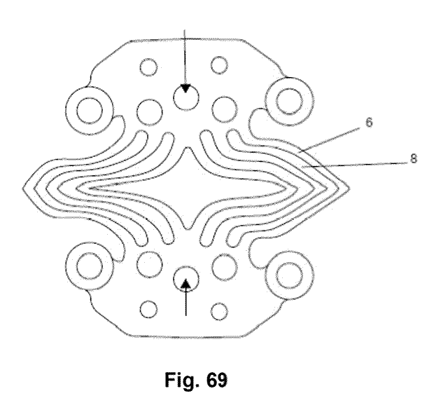

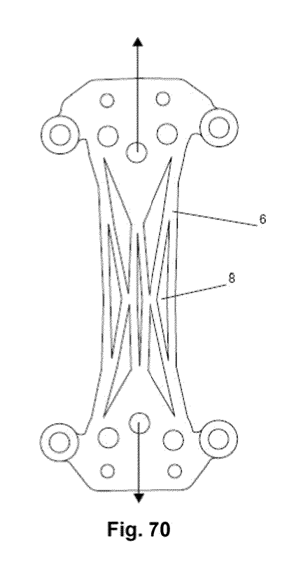

The plate 1 may be deformed before, during, or after it is attached to the spinal column, or a combination thereof. The plate 1 may be deformed in any direction or mode, and to any degree. FIG. 65 illustrates that the plate 1 may be deformed to a short length. FIGS. 66 and 67 illustrate that the plate 1 may be irregularly or asymmetrically deformed. FIG. 68 illustrates that the plate 1 made be deformed in profile, for example to a convex or concave shape, or a combination thereof. FIGS. 70 and 71 illustrate that the plate 1 may be deformed to a long form.

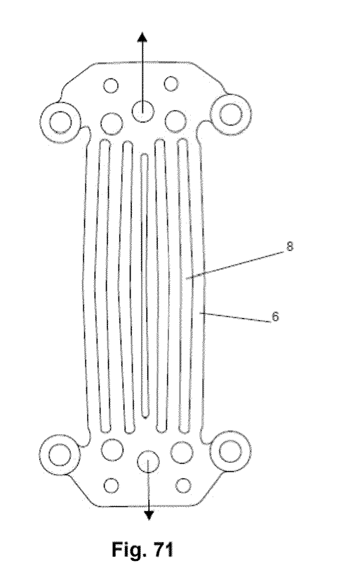

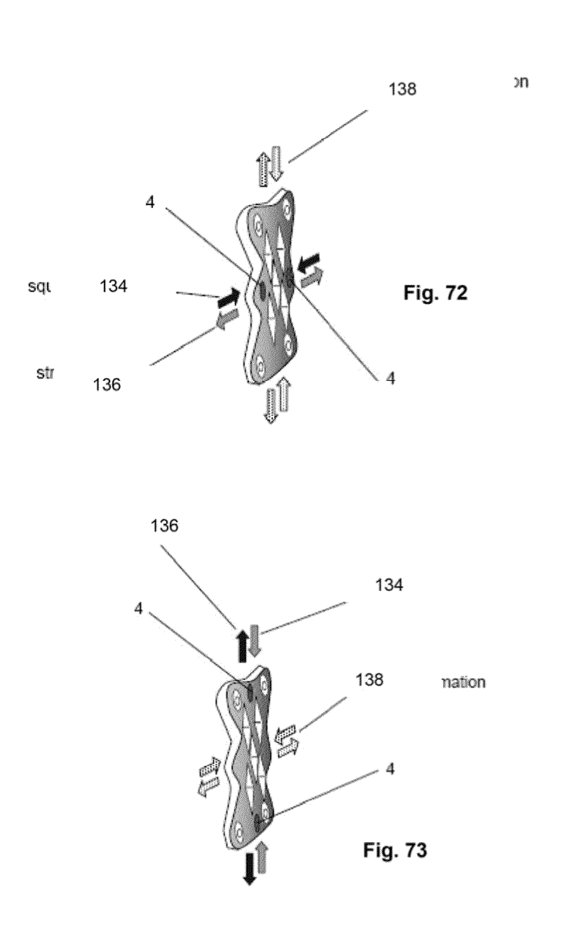

FIGS. 72 and 73 illustrate that the plate 1 can expand or contract in a direction perpendicular to the direction of an applied force. As FIG. 72 illustrates, if a squeeze force 134 is applied to the plate deformation adapters 4 located laterally on the plate 1, the device lengthens (outward facing reaction deformation arrows 138). If a tensile force is applied, the plate shortens (inward facing reaction deformation arrows 138). As FIG. 73 illustrates, if a squeeze force 134 is applied to the plate deformation adapters 4 located on the longitudinal extremes of the plate 1, the plate 1 becomes wider (outward facing reaction deformation arrows). If a stretching force 136 is applied, the plate becomes narrower (inward facing reaction deformation arrows).

The plate 1 may be deformed to between 50 and 200% of its undeployed length, more narrowly between 60 and 150% of its undeployed length, for example 75% or 125%. The plate 1 may be deformed uniformly across its width, or can be deformed more on one side than the other.

The plate 1 can be made from a plastically deformable material, for example a biocompatible metal. Any or all elements of the plate 1 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET)/polyester (e.g., DACRON® from E. I. Du Pont de Nemours and Company, Wilmington, Del.), polypropylene, (PET), polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the plate 1 and/or other devices or apparatuses described herein, can be or have a matrix for cell ingrowth or be used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof.

The elements of the plate 1 and/or other devices or apparatuses described herein and/or the fabric can be filled and/or coated with an agent delivery matrix known to one having ordinary skill in the art and/or a therapeutic and/or diagnostic agent. The agents within these matrices can include radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Spl Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

The plate 1 can be stamped, molded, cast, forged, cut, pressed, sintered, extruded, or a combination thereof. The plates can be laser cut, or non-laser cut. The plate 1 can be laser cut in a partially opened pattern, then the plate 1 can be loaded (e.g., crimped) onto a deployment tool.

The plate 1 can be longitudinally segmented. Multiple plates can be attached leading end to trailing end, and/or a single plate can be severed longitudinally into multiple plates. More than one plate can be deployed in combination or in series. For example, two plates may be screwed together in order to link them and thereby stabilize three or more vertebra.

### Method of Use

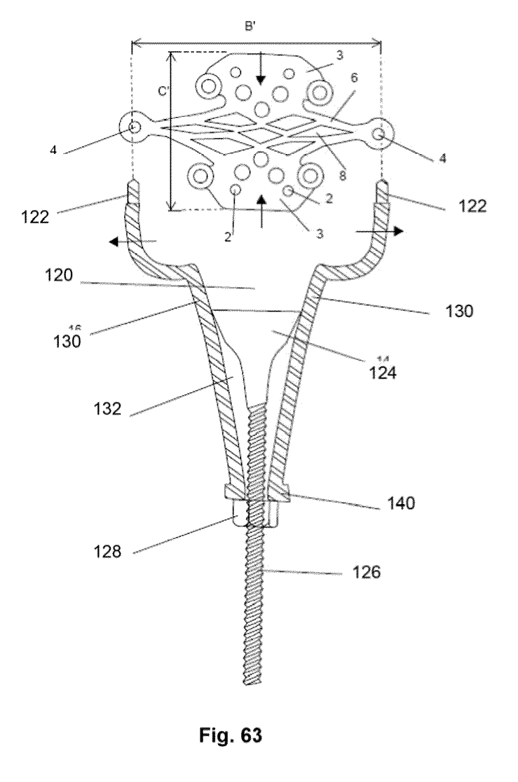

The plate 1 can be deformed either before, during, or after attachment to the vertebra. For example, it may be useful to pre-deform the plate 1 to a shorter height before attaching to two adjacent vertebrae as a part of a decompression, distraction procedure. FIG. 63 illustrates that a plate 1 can be deformed to a shorter height C′, using a deformation tool 120. FIG. 63 illustrates that a plate 1 can be uniformly deformed to a shorter height C′ by inserting the engagement prongs 122 into the plate deformation adapters 4. The deformation tool 120 can then be caused to expand, spreading the engagement prongs 122 apart from one another (as indicated by the opposed black arrows on the deformation tool 120). As FIG. 63 illustrates, the expansion of the deformation tool can be effected, for example, from forcing an expander element 124 to slide and retract into an expansion slot 132, for example due to a tension force applied by a nut 128 on a screw rod 126. As FIG. 63 illustrates, the spreading of the engagement prongs 122 can act through the plate deformation adapters 4 to deform the deformation struts 6, which in turn cause the plate 1 to contract in height to a contracted height C′. As is illustrated by FIG. 63, the contraction in height of the plate 1 can result from the particular arrangement of the deformation struts 6 and the shape of the deformation apertures. As is further illustrated by FIG. 63 the end flanges 3 can retain their original shape after the deformation process, for example, to retain the spacing between the screw-holding sockets.

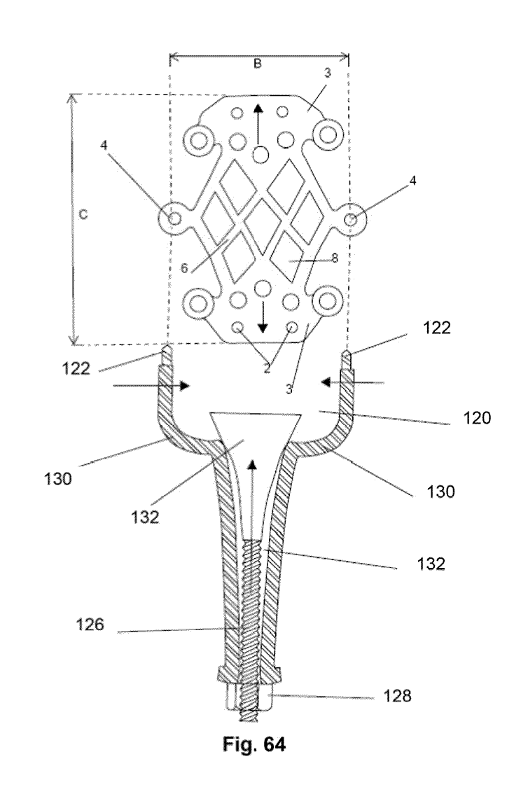

The plates 1 may be transformed more than once, for example pre-operatively deformed, installed in the patient, and then restored to an original or another configuration in situ. FIG. 64 illustrates that the plate can then be re-expanded to a height C by application of a compressive, squeezing force to the plate deformation adapters 4 by means of a deformation tool 120. As is illustrated by FIG. 64, application of a compressive squeezing force to lateral plate deformation adapters 4 can result in an expansion in height in a direction perpendicular to the applied compressive force. This can result from, for example, the action of the deformation struts 6. As FIG. 64 illustrates, the compressive squeezing force can be applied by a deformation tool 120, for example, turning a nut 128 on an expansion rod against an screw rod collet 140, causing the translation of an expander element 124 in an expansion slot 132, thus allowing the pre-loaded elastic arms 130 to squeeze the engagement prongs 122 toward one another.

FIG. 65 illustrates that a deformation force can be applied to more than one set of plate deformation adapters 4 in order to achieve a desired mode of plate deformation. For example, FIG. 65 illustrates that a tensile, stretching force be can be applied to plate deformation adapters 4a in order to result in a perpendicular plate deformation direction E. FIG. 65 further illustrates how a compressive, squeezing force can be applied to plate deformation adapters 4b in order to achieve the same plate deformation E.

FIG. 66 illustrates that a bending asymmetrical deformation can be achieved by more then one mode of applied deformation force. FIG. 66 illustrates that a compressive, squeezing deformation force F can be applied to plate deformation adapters 4 on one side of the plate 1, for example plate deformation adapters 4b, in order to achieve a desired asymmetrical bending. FIG. 66 further illustrates how the same asymmetrical bending deformation can be achieved by applying a tensile, stretching force E on plate deformation adapters 4 located on one side of the plate 1, for example deformation adapters 4a.

FIG. 67 illustrates that an asymmetrical bending deformation can be achieved by the use of two deformation tools together. For example, FIG. 67 illustrates that a first deformation tool 120a can be engaged into two plate deformation adapters 4a on one end of a plate while a second deformation tool 120b can be engaged into a set of deformation adapters 4b on the opposite and of a plate. A twisting force T can be applied to the first deformation tool 120a while an opposite-sense twisting force T′ can simultaneously be applied to the second deformation tool 120b, resulting in a bending plate deformation.

FIG. 68 illustrates that the profile of a plate can be adjusted using two deformation tools together. FIG. 68 illustrates that a first deformation tool 120a can be engaged into a first set of plate deformation adapters 4a on a first end of a plate, while a second deformation tool 120b can be engaged into a second set of plate deformation adapters 4b on the opposite end of the plate. FIG. 68 further illustrates how the first and second deformation tools, 120a and 120b respectively, can then be leveraged toward each other, as indicated by the black arrows, in order to create a bending force across the deformation area 5 of the plate. This bending force can then cause the plate to bend into a curved profile.

After being plastically deformed, the plate can securely retain its deformed configuration during use and throughout its term in service in the patient. FIG. 69 illustrates that the engagement struts 6 can accommodate a compressive deformation while retaining a uniform symmetry. FIG. 69 illustrates that the arrangement of the deformation struts 6 can ensure a uniform and symmetric deformation by plastically deforming in a predetermined manner to accommodate and direct the desired deformation. FIG. 69 illustrates that the deformation struts 6 can deform, for example outward from the center of the plate, folding around one another, to accommodate a compressive deformation force (indicated by the black arrows).

FIGS. 70 and 71 illustrate that a plate can be deformed, for example by stretching or expanding, into a configuration that is longer or taller than its original geometry. FIG. 70 further illustrates how the deformation struts 6 can deform to accommodate a stretching deformation, and thereafter securely retain their deformed configuration. FIGS. 70 and 71 further illustrate how the deformation struts 6 can laterally deform, for example inward toward each other, into the area of the deformation apertures 8, in order to accommodate a stretching deformation mode in response to a stretching deformation force (indicated by the black arrows).

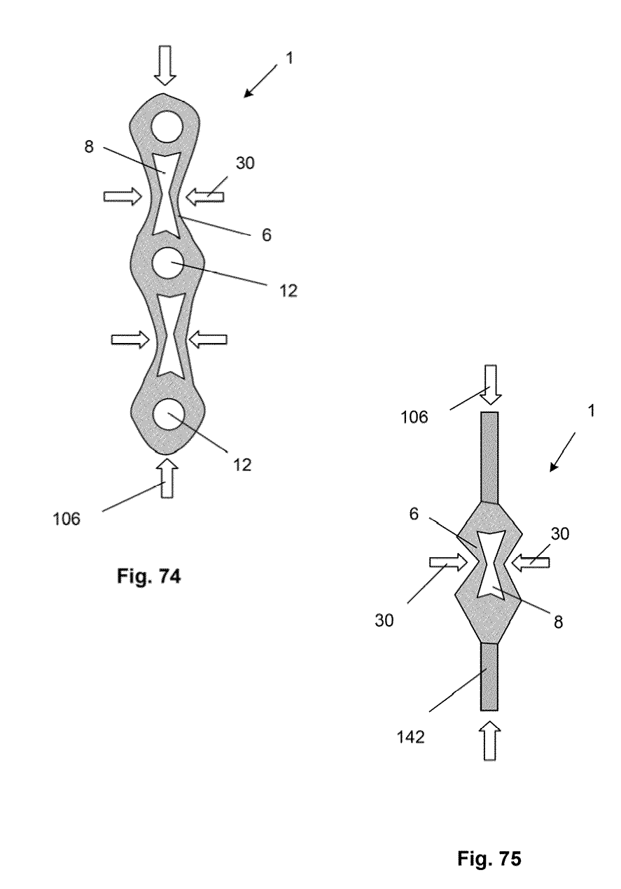

FIG. 74 illustrates that the longitudinal compressive force 106 can cause the struts 6 to deform radially inward (i.e., toward the longitudinal axis of the plate 1). FIG. 75 illustrates that the plate 1 can have head rods 142 extending longitudinally from the plate 1. The compressive force 106 can be applied to the rods 142.

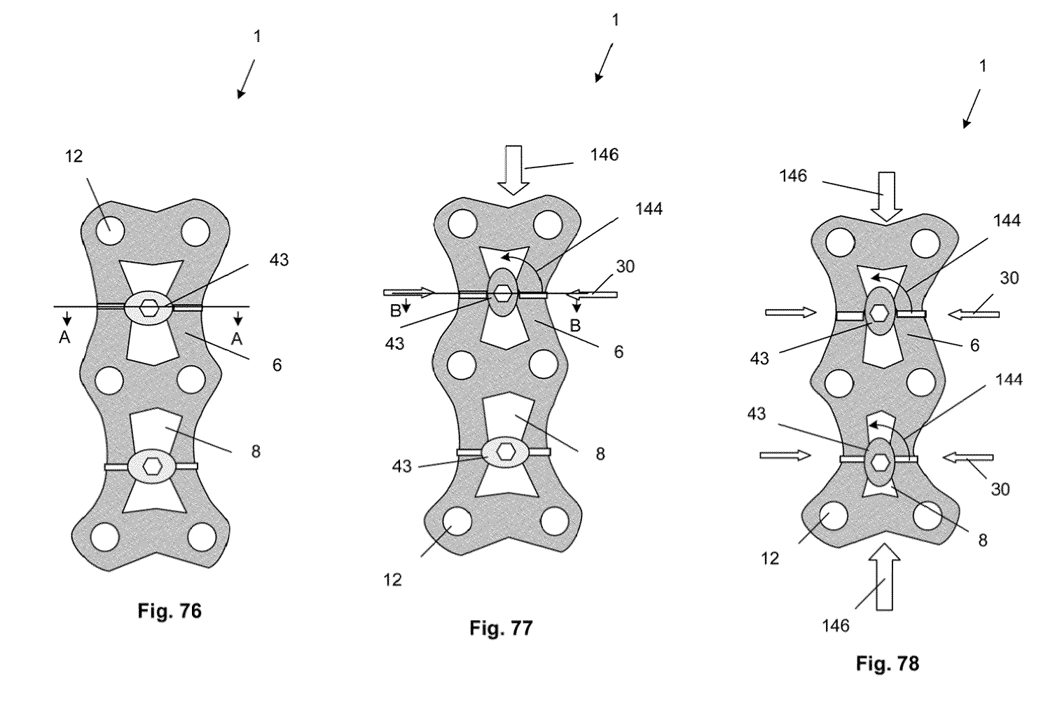

FIG. 76 illustrates that the plate 1 can have two cams 43 installed. The plate 1 can be in a longitudinally extended configuration.

FIG. 77 illustrates that one cam 43 can rotated, as shown by arrow 144. The rotation of the cam can cause the resiliently biased struts 6 adjacent to the cam 43 to deform, as shown by arrows, radially inward toward the center of the plate 1. The deformation of the struts 30 can longitudinally contract, as shown by arrow 146, the half of the plate 1 directly affected by the cam 43.

FIG. 78 illustrates that both cams 43 can be rotated, as shown by arrows 144. The rotation of the cams can cause the resiliently biased struts 6 adjacent to the respective cams to deform, as shown by arrows, radially inward toward the center of the plate 1. The deformation of the struts 30 can longitudinally contract, as shown by arrow 146, the entire length of the plate 1.

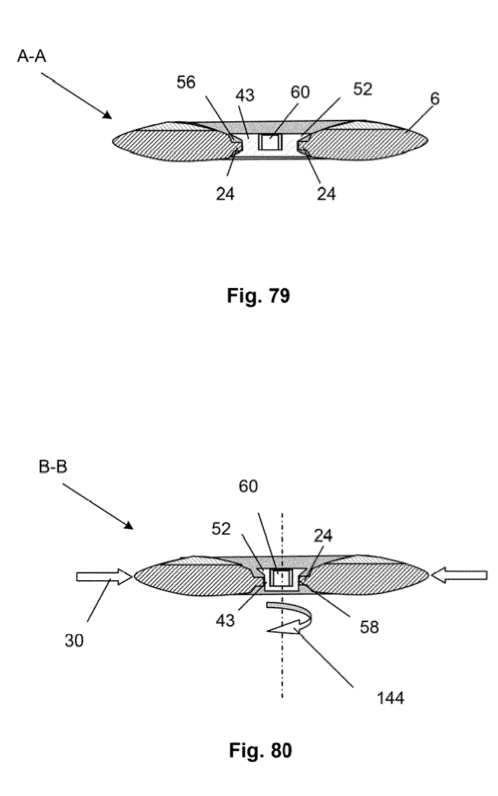

FIG. 79 illustrates that cam 43 can be engaged between the stops 24. The cam 43 can resiliently deform the struts 6 radially outward. FIG. 80 illustrates that the cam can be rotated, as shown by arrow 144. The cam 43 can be rotated so the short axis of the cam 43 is between the stops 24. The struts 6 can return to the unbiased configuration of the struts 6, as shown by the radially inward strut deformation arrows 30. The cam lip 52 can be above and/or below the cam side slot 58.

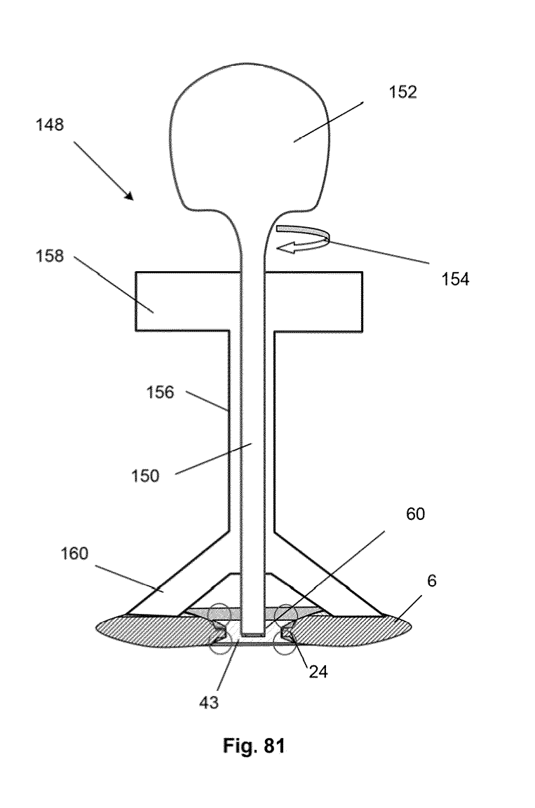

FIG. 81 illustrates that a cam adjustment tool 148 can be used to rotate the cams 43 in the plate 1. The cam adjustment tool 148 can have an inner torque rod 150 that can be inserted into the cam attachment port 60 to transmit torque to the cam 43. The inner torque rod 150 can extend to a torque handle 152. A torque can be applied to the torque handle 152 to rotate, as shown by arrow 154, the inner rod.

The cam adjustment tool 148 can have an outer stability shaft 156. The inner torque rod 150 can be rotationally substantially contained within the outer stability shaft 156. The outer stability shaft 156 can have a radially extending stability handle 158 and one or more stability legs 160 (or a stability cone). The stability legs 160 can rest on the plate or surrounding material (e.g., tissue, prep table). The stability handle 158 can be held steady during rotation of the cam.

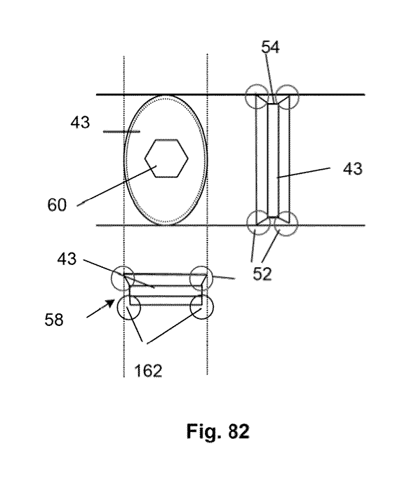

FIG. 82 illustrates that the cam 43 can have no bottom lip 162 along the short axis of the cam 43, adjacent to the cam side slot 58. The minimum outer diameter of the cam lip 52 can be larger than the distance between laterally adjacent stops when the plate is in a relaxed, unbiased configuration.

The cams 43 or other screws or distracting elements used in lieu of cams 43 can be made from any material disclosed herein such as Ti, Steel, Niti, one or more polymers (e.g., Delran, PET, Nylon, PEEK, PEKK, PTFE), or combinations thereof.

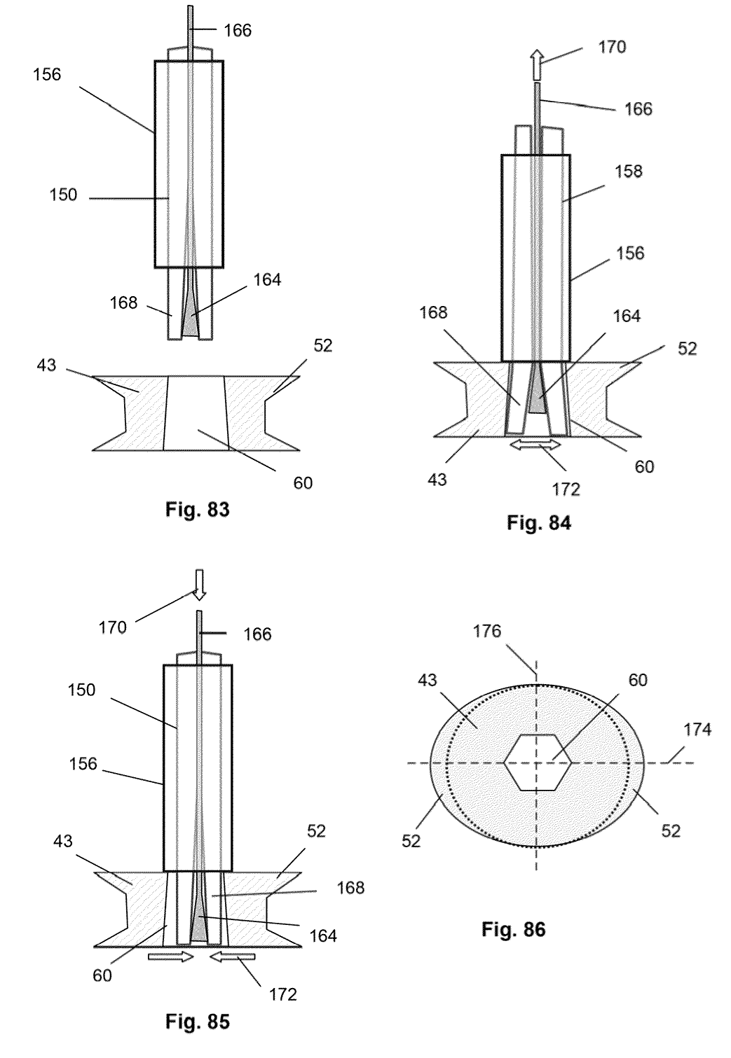

FIG. 83 illustrates that the inner torque rod 150 can have multiple cam engaging members surrounding by a slidably attached cone-shaped expander head 164. The expander head 164 can be the distal end of a tool expander 166. The tool expander 166 and expander head 164 can be a slidable shaft or a rotatable screw. The expander head 164 and cam engaging members 168 can extend out of the outer stability shaft 156.

The cam attachment port 60 can have a round or hexagonal conical configuration.

FIG. 84 illustrates that the expander head 164 and cam engaging members 168 can be inserted into the cam attachment port 60. The tool expander 166 can be translated (or rotated) away, as shown by arrow 170, from the cam 43. The expander head 164 can then force the cam engaging members 168 radially outward, expanding the tool (i.e., the cam engaging members 168), as shown by arrows 172, to fit the cam attachment port 60. The inner torque rod 150 can then be rotated. The torque from the inner torque rod 150 can be transmitted to the cam 43 via the cam engaging members 168.

FIG. 85 illustrates that when the tool is to be removed from the cam 43, the tool expander 166 can be translated (or rotated) toward, as shown by arrow 170, the cam 43. The cam engaging members 168 can then resiliently relax, as shown by arrows 172, to a radially contracted configuration. The tool can then be removed from the cam 43.

FIG. 86 illustrates that the cam lip 52 can be largest along the cam major axis 174 (i.e., cam long axis). The cam lip can be absent along the cam minor axis 176 (i.e., cam short axis).

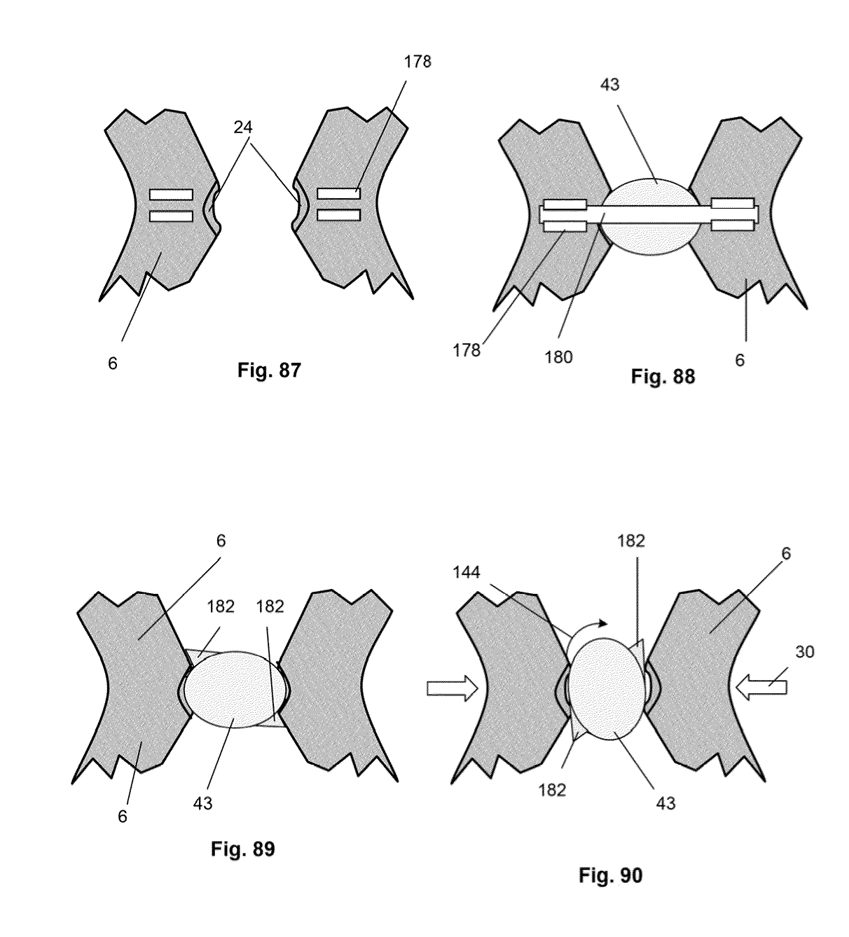

FIG. 87 illustrates that the struts 6 adjacent to the stops 24 can have one or more engagers 178. The engagers 178 can be individual, in pairs, or larger sets adjacent to each stop 24. The engagers 178 can be parallel to each other. The engagers 178 can be rails, grooves, threaded ports, magnets, or combinations thereof.

FIG. 88 illustrates that a cam 43 can be between the stops 24. A locking bar 180 can be placed over the cam 43. The locking bar 180 can be help to the struts 6 be the engagers 178. For example, the locking bar 180 can be slid under the engagers 178.

FIG. 89 illustrates that the cam 43 can have one, two, or more rotation limiters 182. The rotation limiters 182 can be triangular or other shaped extensions from the cam 43. The rotation limiters 182 can be 180° apart on the cam 43. The rotation limiters 182 can interference fit with the struts 6, for example to restrain rotation of the strut 6 to a sub −180° range and provide full-stops (e.g., for tactile feedback).

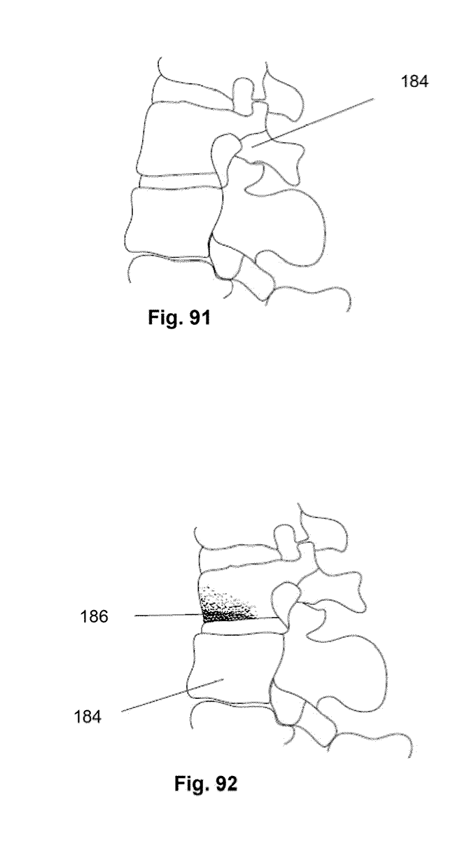

FIG. 90 illustrates that the rotation limiter 182 can abut the strut 6 when the cam is rotated, as shown by arrow 144, to the extent allowed by the rotation limiter 182.

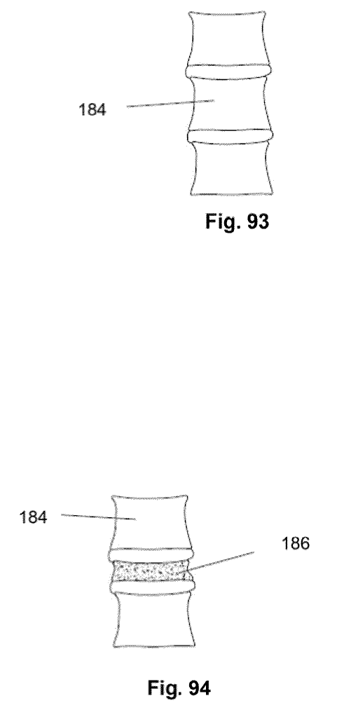

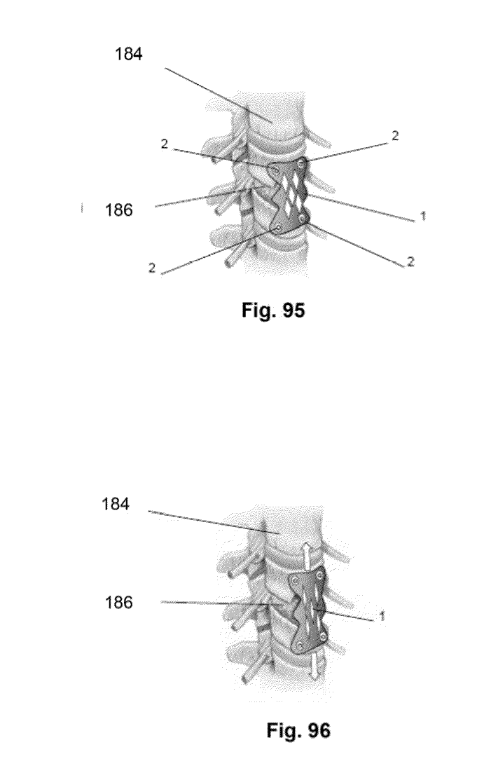

The plates can be employed to treat compression-type injuries to the vertebra. FIGS. 91 and 92 illustrate a spinal column 184 before and after a compressive-type vertebral injury, respectively. FIGS. 93 and 94 provide an anterior view of the same spinal column 184 before and after suffering from the same vertebral injury, respectively. FIG. 95 illustrates that a plate 1 can be attached to two adjacent vertebra at screw holding sockets 2, bracketing the injury site 186, FIG. 96 illustrates how the plate 1 can then be transformed, for example, with a deformation tool 120, in order to decompress the injury site 186 (the plate transformation direction are indicated by the arrows).

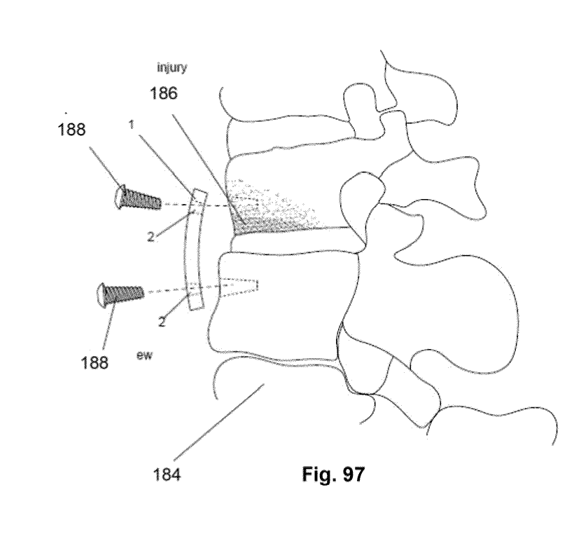

FIG. 97 illustrates that the plate 1 can be placed and located, either in conjunction with preliminary transformation or not, so that the screw holding sockets 2 span two adjacent vertebrae and the injury site 186, and then positioned to facilitate attachment by use of bone screws 188.

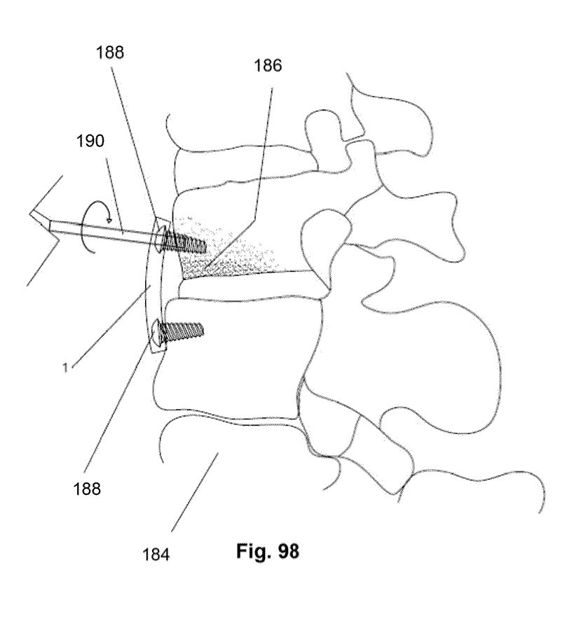

FIG. 98 illustrates that after being properly positioned, the plate 1 can then be secured to the vertebra, for example by screwing in the bone screws 188 with a tool 190.

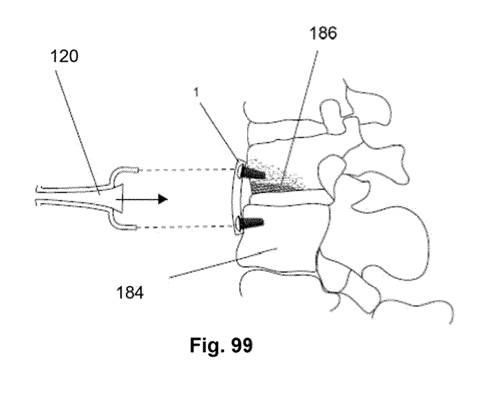

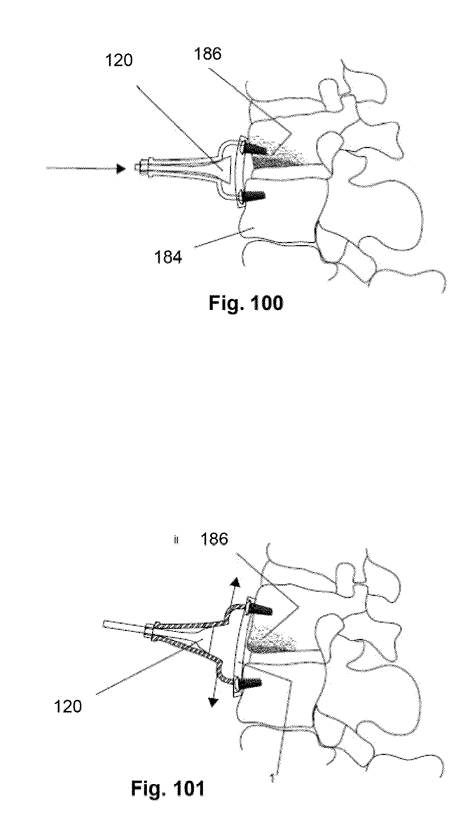

FIGS. 99 and 100 illustrate that a deformation tool 120 can be engaged into a plate 1 after it has been screwed into two adjacent vertebrae on an injured spinal column 184. FIG. 101 illustrates how the deformation tool 120 can then be expanded (indicated by the arrows), thereby deforming the plate 1, distracting the two vertebrae, and decompressing the injury site 186. FIG. 102 illustrates that the transformed plate 1 can securely maintain the decompressed spinal configuration 192 after the removal of the deformation tool 120.

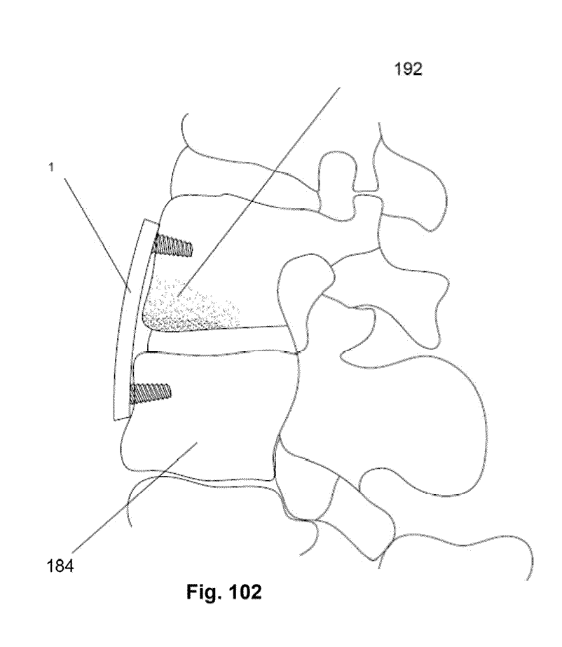

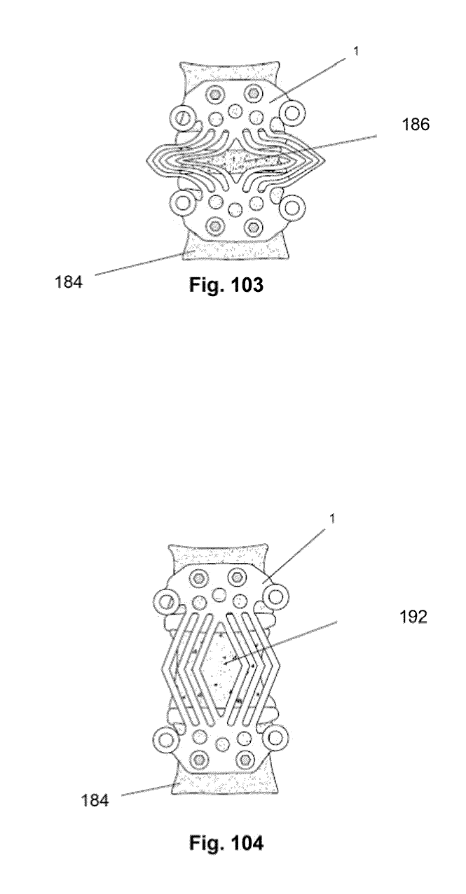

FIG. 103 illustrates from an anterior view that a pre-compressed plate 1 can be attached to two adjacent vertebrae bracketing a compression-type injury site 186. FIG. 104 illustrates that the plate 1 can be expanded to decompress the injury site 186 and thereafter securely maintain the decompressed configuration 192 after the removal of the deformation tool 120.

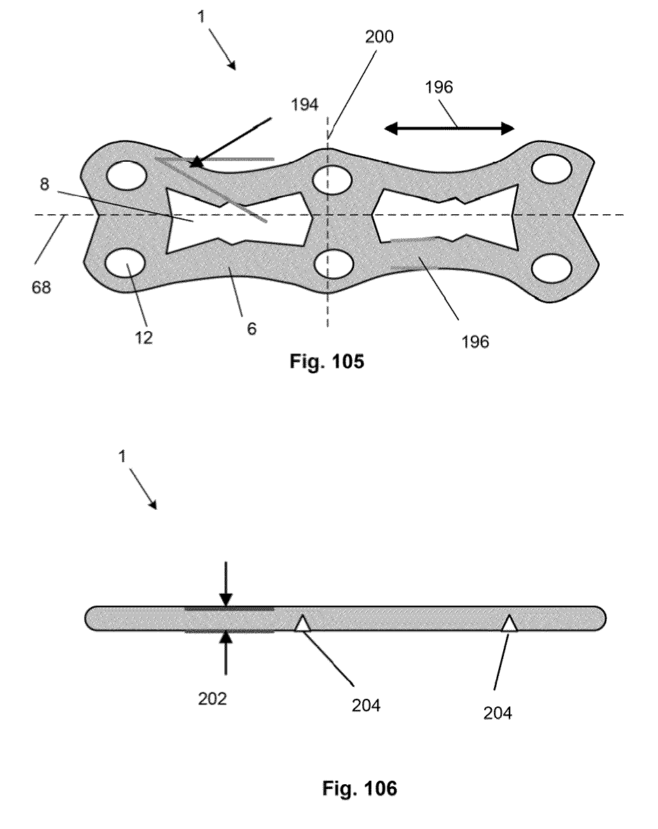

FIG. 105 illustrates that the struts 6 can have strut angles 194 relative to (a parallel from) the longitudinal axis 68. The struts 6 can have strut widths 196. The struts can have strut lengths 198. FIG. 106 illustrates that the struts 6 can have strut thicknesses 202.

The plate 1 can have hinge points 204. The hinge points 204 can enable rotation of the plate 1 with respect to the transverse axis 200.

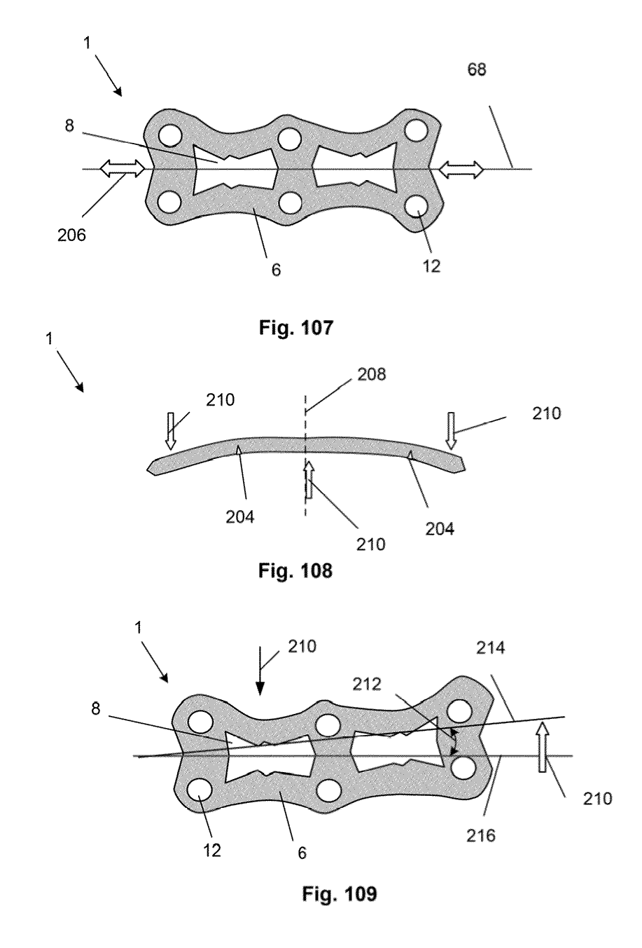

FIG. 107 illustrates that the plate 1 can sustain compressive and tensile forces 206 during use. The dimensions of the plate 1 can be changed to satisfy load requirements. For example, reducing strut width will reduce the force required to shorten the plate 1.

FIGS. 108 and 109 illustrate that reducing the strut thickness 202 can reduce the force needed to bend the plate 1. FIG. 108 illustrates that the plate 1 can be bent about the traverse axis. Bending forces, as shown by arrows 210, can be applied in a first direction (e.g., toward the bone or away from the bone) and in a second direction (e.g., the opposite of the other bending forces). The opposed bending forces can create a torque against the plate 1 about the hinge points 204. The plate 1 can flex, bend or otherwise rotate in the transverse direction at the hinge points 204.

FIG. 109 illustrates that the plate 1 can be bent by the bending forces, as shown by arrows 210, about the vertical axis 208. (Note that the vertical axis 208 is approximately horizontal when the plate 1 is implanted and the patient is standing up vertically.) The plate 1 can bend to a bending angle 212 measured from the original longitudinal axis 216 to a bent longitudinal axis 214.

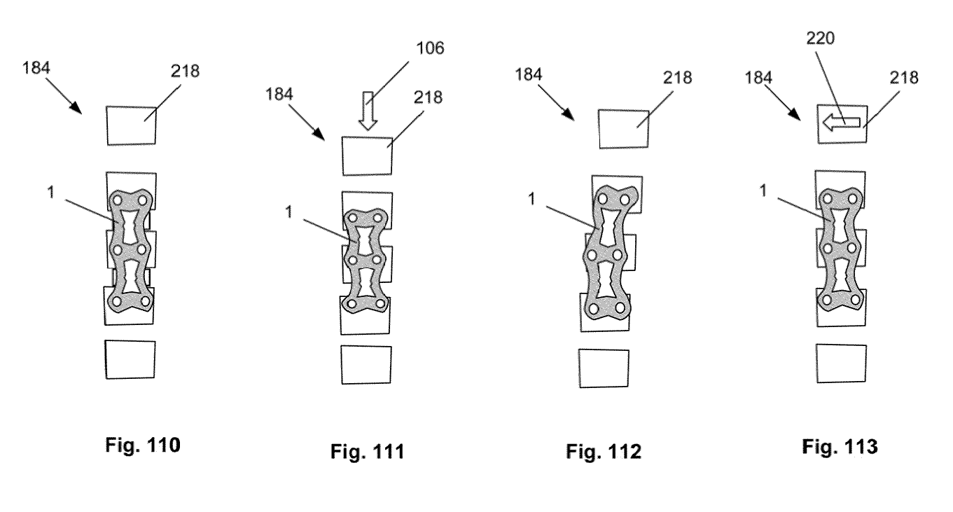

FIG. 110 illustrates that the plate 1 can be attached to vertebrae 218. The vertebrae 218 can be axially stretched apart, longitudinally expanding the plate 1. FIG. 111 illustrates that the plate 1 can apply a compressive force, as shown by arrow 106, to the vertebrae 218 (i.e., via the bone screws, fixation screws 224 or pins (not shown) through the attachment holes). The vertebra 218 and the plate 1 can shorten longitudinally.

FIG. 112 illustrates that the vertebrae 218 can be axially misaligned. The plate 1 can be fixed to the vertebrae 218 in a configuration to match the configuration of the vertebrae 218 (e.g., rotated). The plate 1 can then be set to be in rotational tension or compression. FIG. 113 illustrates that the tension or compression of the plate 1 can then exert a rotation torque, as shown by arrow 220, on the vertebrae 218. The vertebrae 218 and plate 1 can then become axially aligned.

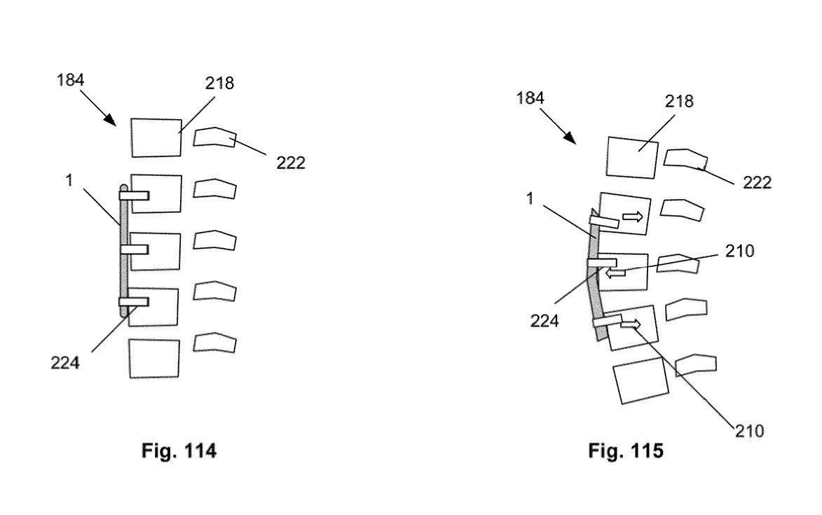

FIG. 114 illustrates that the vertebrae 218 can be axially misaligned in a different plane than that shown in FIG. 112. The plate 1 can be fixed to the vertebrae 218 in a configuration to match the configuration of the vertebrae 218 (e.g., flat, whereas it should be curved under physiological conditions). The plate 1 can then be set to be in rotational tension or compression. FIG. 115 illustrates that the tension or compression of the plate 1 can then exert bending forces, as shown by arrows 210, on the vertebrae 218. The vertebrae 218 and plate 1 can then become axially aligned.

The device can be adjustable before or after implantation (e.g., deployment, delivery, insertion). The adjustments can include length, width, height, pitch, yaw, or combinations thereof.

The device can be designed to change shape once implanted.

The device can act like a spring creating compaction and stability.

The device can have moving surfaces or no moving surfaces (e.g., screws in plate, screw through bone, slides, plate on bone).

As the vertebral bodies settle, the graft can tighten, and the plate can shorten, this can have no effect on disc space above and below the fusion.

The stops can have curved configurations, for example, forming saddles to seat the cams. The stops can be coated or padded with a material, for example a polymer such as PTFE (e.g., Teflon).

The plate is referred to herein as the device.

It is apparent to one skilled in the art that various changes and modifications can be made to this disclosure, and equivalents employed, without departing from the spirit and scope of the invention. Elements shown with any embodiment are exemplary for the specific embodiment and can be used in combination with or otherwise on other embodiments within this disclosure.

We claim:

1. A bone support device for adjustably providing support to a spinal column, comprising:
a first component and a second component, wherein the first component translatably interfaces with the second component; and
wherein the first component comprises a first component head, a first flexible leg extending from the first component head, and a second leg extending from the first component head, wherein the space between the first flexible leg and the second leg defines a first arm slot; and
wherein the second component comprises a second component head and a first arm extending from the second component head; and
wherein the first arm is received by the first arm slot; and wherein the first arm is configured to slidably engage the first flexible leg and the second leg; and
wherein the first flexible leg has a first flexible leg terminal end, and wherein the second leg has a second leg terminal end.

2. The device of claim 1, wherein the first component has a first central longitudinal axis, and wherein the second component has a second central longitudinal axis, and wherein the first component further comprises a first attachment hole and a second attachment hole symmetrically offset from the first attachment hole with respect to the first central longitudinal axis, and wherein the second component further comprises a third attachment hole and a fourth attachment hole symmetrically offset from the second attachment hole with respect to the second longitudinal axis.

3. The device of claim 2, wherein an extension of the first central longitudinal axis is substantially the same as the second central longitudinal axis.

4. The device of claim 1, wherein the first component is configured to be unidirectionally translatably engaged with the second component.

5. The device of claim 1, wherein the first component is configured to be bidirectionally slidably received by the second component.

6. The device of claim 1, wherein the first component comprises a third flexible leg extending from the first component head, and a fourth leg extending from the first component head, wherein the space between the third flexible leg and the fourth leg defines a second arm slot; and wherein the second component comprises a second arm extending from the second component head; and wherein the second arm is received by the second arm slot; and wherein the second arm is configured to slidably engage the third flexible leg and the fourth leg; and wherein the third flexible leg has a third flexible leg terminal end, and wherein the fourth leg has a fourth leg terminal end.

7. The device of claim 1, wherein the second component comprises a third flexible leg extending from the second component head, and a fourth leg extending from the second component head, wherein the space between the third flexible leg and the fourth leg defines a second arm slot; and wherein the first component comprises a second arm extending from the first component head; and wherein the second arm is received by the second arm slot; and wherein the second arm is configured to slidably engage the third flexible leg and the fourth leg; and wherein the third flexible leg has a third flexible leg terminal end, and wherein the fourth leg has a fourth leg terminal end.

8. The device of claim 1, wherein the first arm comprises a first arm tooth extending toward the first flexible leg, and wherein the first flexible leg comprises a first flexible leg tooth extending toward the first arm.

9. The device of claim 1, wherein the first arm has a first arm terminal end.

10. The device of claim 1, wherein the first component and the second component have a first longitudinal axis, and wherein the first component and the second component are configured to substantially have a non-infinite radius of curvature relative to a second longitudinal axis parallel to the first longitudinal axis offset in the direction of the spinal column.

11. The device of claim 1, further comprising a vertebral attachment element, wherein the vertebral attachment element comprises a screw-holding socket configured to receive and rigidly attach to a mounting.

12. The device of claim 11, wherein the plate deformation adapter comprises a socket adapted to receive a deformation tool.

13. The device of claim 12, further comprising a deformation tool removably connectable to the plate, adapted to attach to the plate deformation socket.

14. The device of claim 13, wherein the plate defines one or more deformation apertures adapted to direct the mode of plate deformation in specific deformation directions in response to specific deformation forces applied to the plate.

15. The device of claim 14, wherein the deformation directions are perpendicular to the deformation forces.

16. A method of performing a spinal treatment comprising:
preparing a spinal column to receive a spinal stabilization plate,
attaching the plate to a spinal column having a first bone and a second bone, wherein the plate comprises a first plate section slidably received in a second plate section; and
slidably adjusting the first plate section with respect to the second plate section, wherein the first plate comprises first plate head, a first flexible leg extending from the first plate head, and a second leg extending from the first plate head, wherein the space between the first flexible leg and the second leg defines a first arm slot, and wherein the second plate comprises a second plate head and a first arm extending from the second plate head, wherein the first flexible leg has a first flexible leg terminal end, and wherein the second leg has a second leg terminal end, and wherein the first arm has a first arm terminal end, and wherein adjusting comprises sliding the first arm in the first arm slot; and
wherein attaching the plate comprises attaching the first plate section to the first bone and attaching the second plate section to the second bone.

17. The method of claim 16, wherein attaching the spinal stabilization plate to a spinal column comprises:
positioning the plate over a first vertebra so that a first screw holding socket is aligned with a first suitable bone screw attachment point on the first vertebra, partially securing the first screw holding socket to the first suitable bone screw attachment point on the first vertebra with a bone fixation screw, positioning a second screw holding socket of the spinal stabilization plate into alignment with a second suitable bone screw attachment point on a second vertebra, securing the second screw holding socket to the second suitable bone screw attachment point with a bone screw, and fully securing the first screw holding socket to the first suitable bone screw attachment point.

18. The method of claim 17, further comprising the step of transforming the plate by plastically deforming the plate.

19. The method of claim 18, wherein the transforming the plate by resiliently deforming the plate comprises: attaching a deformation tool to attachment points on the plate, manipulating the deformation tool to deform the plate into a desired configuration, and removing the deformation tool from the plate.
